(19) United States
(12) Patent Application Publication
(10) Pub. No.: US 2002/0106348 A1
(43) Pub. Date: Aug. 8, 2002

(54) Cancer therapeutics involving the administration of 2-methoxyestradiol and an agent that increases intracellular superoxide anion

(76) Inventors: Peng Huang, Houston, TX (US); William K. Plunkett, Houston, TX (US); Li Feng, Sugar Land, TX (US)

Correspondence Address: FULBRIGHT & JAWORSKI L.L.P., A REGISTERED LIMITED LIABILITY PARTNERSHIP, 600 CONGRESS AVENUE, SUITE 2400, AUSTIN, TX, 78701, US

(21) Appl. No.: 09/899,807

(22) Filed: Jul. 5, 2001

Related U.S. Application Data
(60) Provisional application No. 60/217,589, filed on Jul. 12, 2000.

(51) Int. Cl. A61K 38/19; A61K 38/08; A61K 31/704; A61K 31/56

(52) U.S. Cl. 424/85.1; 514/8; 514/34; 514/72; 514/182

(57) ABSTRACT

The instant invention discloses methods and compositions for the treatment of cancer. The invention relates methods and compositions for specifically targeting free radical accumulation as a means of preferentially eliminating neoplastic cells. The combination of an SOD inhibitor (2-methoxyestrdiol) with free radical-producing agents results in a means of eliminating tumor cells through the accumulation of intracellular superoxide anion.

### BACKGROUND OF THE INVENTION

This application claims priority to provisional application No. 60/217,589 filed Jul. 12, 2000, herein incorporated by reference.

### FIELD OF THE INVENTION

The present invention relates generally to the field of cancer therapeutics. More particularly, it concerns compositions and methods for increasing intracellular O₂⁻ while inhibiting the activity of superoxide dismutase.

### DESCRIPTION OF RELATED ART

Superoxide anion, O₂⁻, is a toxic reactive oxygen intermediate produced by the transfer of a single electron to O₂. Superoxide anion is formed in the process of cellular respiration, specifically during oxidative phosphorylation. While oxidative pathways have evolved to minimize O₂⁻ production, a small amount of superoxide anion is unavoidably formed during the metabolic reduction of oxygen.

Reactive oxygen intermediates have been implicated in a number of human degenerative processes, diseases and syndromes, including the following: mutagenesis, cell transformation and cancer; atherosclerosis, arteriosclerosis, heart attacks, strokes and ischaemia/reperfusion injury; chronic inflammatory diseases, such as rheumatoid arthritis, lupus erythematosus and psoriatic arthritis; acute inflammatory problems, such as wound healing; photo-oxidative stresses to the eye, such as cataract; central-nervous-system disorders, such as certain forms of familial amyotrophic lateral sclerosis, certain glutathione peroxidase-linked adolescent seizures, Parkinson's disease and Alzheimer's dementia; and a wide variety of age-related disorders, possibly including factors underlying the aging process itself. Reactive oxygen intermediates are known to produce a variety of pathological changes through lipid peroxidation and DNA damage. The common pathway of tissue injury mediated by these toxic oxygen metabolites involves the destruction of membranes, proteins, and nucleic acids.

Antioxidant enzymes protect cells from the toxic effects of high concentrations of reactive oxygen species generated during cellular metabolism. Superoxide dismutases (SOD) are metalloenzymes that catalyze the dismutation of superoxide ion into oxygen and hydrogen peroxide:

O₂⁻+O₂⁻+2H⁺ →H₂O₂+O₂

The enzymes scavenge superoxide anion and act as a primary defense system against oxidative stress in body. Three classes of SODs have been described, each characterized by the catalytic metal at the active site, namely, Cu/Zn-SOD, Mn-SOD, and Fe-SOD. Cu/Zn enzymes are found primarily in eukaryotes, Fe-SOD is found mainly in prokaryotes and Mn-SOD crosses the entire range from prokaryotes to eukaryotes. The CuZn-SOD is localized in the cytosol and nucleus, while Mn-SOD is located within the mitochondrial matrix. It has been widely recognized that such enzymes provide a defense system that is essential for the survival of aerobic organisms.

Carcinogens and tumor promoters are known to decrease the cellular activity of superoxide dismutase. Consequently, many neoplastic cells have reduced superoxide dismutase activity. Tumor cells nevertheless, carry out normal oxidative pathways and thus accumulate reactive oxygen intermediates at a rate at least equal to normal cells.

The critical function of the dismutation of O₂⁻ makes SOD an attractive target for pharmacological intervention. Although several small molecules, including cyanide ion (CN⁻), hydroxyl ion (OH⁻), and azide ion (N₃⁻), inhibit SOD by competing with O₂⁻ at the catalytic site, these chemicals are highly toxic and their potential for cancer therapy is limited.

### SUMMARY OF THE INVENTION

The instant invention addresses a noted deficiency in the art by providing therapeutic compositions and methods for the treatment of cancer. The invention discloses that 2-methoxyestradiol inhibits SOD and compromises the cell's ability to eliminate superoxide anion, and that the combination of 2-methoxyestradiol with an agent or agents that increases reactive oxygen intermediates within a cell results in an enhanced ability to kill cells, specifically cancer cells. While both classes of agents may be used independently as cancer therapeutics, the instant invention discloses that the mechanism-based combination of compounds produces a synergistic effect that dramatically increases the tumoricidal and/or anti-neoplastic efficacy of each compound.

In one embodiment, the instant invention therefore comprises a method of killing a cell comprising contacting the cell with a first composition comprising an agent that increases intracellular O₂⁻ and with a second composition comprising 2-methoxyestradiol. Alternate embodiments of this method contemplate that the two compositions may be provided substantially concurrently or the 2-methoxyestradiol may be delivered prior to or subsequent to administration of the agent that increases intracellular O₂⁻. In a further embodiment, the two compositions may be combined in a single formulation.

A variety of chemical compounds and physical modalities are known to increase the intracellular concentration of reactive oxygen intermediates, i.e., O₂⁻. In a particular embodiment of the invention, the compound administered to increase intracellular O₂⁻ will be rotenone, tumor necrosis factor-alpha, cisplatin, bleomycin, an arsenate (i.e. arsenic trioxide), a retinoic acid derivative such as all-trans retinoic acid, or an anthracycline. Where the compound is an anthracycline, it may be doxorubicin, daunorubicin, epirubcin or daunomycin. A physical modality administered to increase intracellular O₂⁻ will be heat (hyperthermia), ultraviolet rays, X-rays or γ-rays.

It is contemplated that the method of the instant invention may be used in a variety of applications to kill a specific cell. The instant invention is equally applicable in either an in vivo or in vitro environment. In another embodiment of the invention, the cell to be killed is a cancer cell. In alternate aspects of the invention, the cancer cell is derived from a solid tumor or is a leukemia cell. In a still further aspect, the cell is a human cell.

An alternate embodiment of the invention relates a method of treating cancer comprising administering to a host a first composition comprising 2-methoxyestradiol and a second composition comprising an agent that increases intracellular O₂⁻.

The invention is further contemplated to encompass a composition comprising 2-methoxyestradiol and an agent capable of increasing the intracellular concentration of O₂⁻. In a specific embodiment of this aspect of the invention, the compound administered to increase intracellular O₂⁻ will be rotenone, tumor necrosis factor-alpha, cisplatin, bleomycin, an arsenate (i.e. arsenic trioxide), a retinoic acid derivative such as all-trans retinoic acid, or an anthracycline. Where the compound is an anthracycline, it may be doxorubicin, daunorubicin, epirubcin or daunomycin. A physical agent administered to increase intracellular O₂⁻ will be heat (hyperthennia), ultraviolet rays, X-rays or γ-rays.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Chemical Structure of 2-methoxyestradiol.

FIG. 2: Chemical Structure of rotenone.

FIG. 3: Effect of 2-methoxyestradiol on Survival of K562 Cells (MTT, 72 h).

FIG. 4: Effect of 2-methoxyestradiol on the Survival of Raji Cells (MTT, 72 h).

FIG. 5: Induction of Apoptosis by 2-methoxyestradiol in Primary Leukemia Cells.

FIG. 6: Effect of 2-methoxyestradiol (M) and Fludarabine (F) on the Survival of Primary CLL cells (Patient no. 1, MTT, 72 h).

FIG. 7: Effect of 2-methoxyestradiol (M) and Fludarabine (F) on the Survival of Primary CLL cells (Patient no. 2, MTT, 96 h).

FIG. 8: Effect of 2-methoxyestradiol (M) and Fludarabine (F) on the Survival of Primary CLL cells (Patient no. 3, MTT, 96 h).

FIG. 9: Effect of 2-methoxyestradiol on the Growth of Breast Cancer Cells (MTT, 72 h).

FIG. 10 Effect of 2-methoxyestradiol on the Growth of Lung Cancer Cells (MTT, 72 h).

FIG. 11 Identification of SOD as a Target of 2-methoxyestradiol by Human cDNA Expression Array I.

FIG. 12: Effect of 2-methoxyestradiol (1 μMM) on SOD Protein Level in ML-1 and HL-60 Cells.

FIG. 13: Inhibition of H₂O₂ Production by 2-methoxyestradiol in Whole Cells.

FIG. 14: Loss of Mitochondria Membrane Potential Induced by 2-methoxyestradiol (2 μM) in HL-60 Cells.

FIG. 15: Release of Cytochrome c to Cytosol (HL-60, 2-methoxyestradiol, 2 μM).

FIG. 16: Activation of JNK is not Essential for Apoptosis Induction by 2-methoxyestradiol (2-methoxyestradiol, 1 μM; FSK, 10 μM, 24 h).

FIGS. 17A, 17B, 17C, 17D and 17E: Selective cytotoxicity of 2-ME against human leukemia cells.

FIG. 17A: DNA fragmentation in ML-1 and HL-60 cells incubated with 1 μM 2-ME.

FIG. 17B: DNA fragmentation in normal lymphocytes. Lane 1-4, lymphocytes incubated with 0, 3, 10, and 30 μM 2-ME for 48 h; lanes 5-8, PHA-stimulated (5 μg/ml) lymphocytes incubated with 0, 3, 10, and 30 μM 2-ME for 48 h.

FIG. 17C: Effect of 2-ME on normal and PHA-stimulated lymphocytes (MTT assay, 72 h).

FIG. 17D: Effect 2-ME on primary leukemia cells and normal lymphocytes (MTT assay).

FIG. 17E: Comparison of 2-ME activity in CLL cells (n=31) and normal lymphocytes (n=9).

FIGS. 18A, 18B, 18C and 18D: Effect of 2-ME on SOD expression and free radical metabolism in leukemia cells.

FIG. 18A: RT-PCR analysis of SOD1 mRNA in 2-ME-treated cells.

FIG. 18B: Effect of 2-ME (2 μM) on SOD1 and SOD2 protein expression in ML-1 and HL-60 cells.

FIG. 18C: O₂⁻ accumulation in ML-1 cells treated with 1 μM 2-ME for 5 h. The dotted lines indicate the mean fluorescence intensity.

FIG. 18D: Accumulation of O₂⁻ in primary CLL cells treated with 10 μM 2-ME for 24 h.

FIGS. 19A, 19B, 19C and 19D: Inhibition of SOD by 2-ME.

FIG. 19A: In vitro assay of SOD1 activity.

FIG. 19B: Effect of 2-ME on human and bovine CuZnSOD and E. coli MnSOD.

FIG. 19C Effect of 2-ME on xanthine oxidase.

FIG. 19D: Effect of 2-ME on human DNA polymerase α and bovine alkaline phosphatase (measured by removal of 5′-phosphate from [¹⁴C]GMP).

FIG. 20: Structure of estrogen derivatives, inhibition of CuZnSOD, and induction of apoptosis in HL-60 cells. The degree of SOD inhibition: (−), less then 25% inhibition; (+), 25-50%; (++), 50-75%; (+++), 75-100%. Lane (−), control; lanes 1-5, cells treated with the respectively numbered compounds.

FIGS. 21A, 21B, 21C 21D, 21E, and 21F:

FIG. 21A: Effect of SOD1 overexpression on 2-ME-induced apoptosis. Lane 1, control A2008 cells; lanes 2-3, transduction with Ad.CuZnSOD for 24 and 48 h; lane 4, control vector (48 h). 10 μM 2-ME was added 24 h after transduction and incubated for another 48 h before DNA fragmentation assay.

FIGS. 21B-C: Effect of ectopic expression of SOD1 or SOD2 on the survival of A2008 cells (MTT assay) and H1299 cells (colony formation).

FIG. 21D: SOD antisense S-oligos enhanced the activity of 2-ME. A2008 cells were incubated with S-oligos against SOD1 (SEQ ID NO: 1, 5′-ACGCACACGGCCTTCGTCGCCATAACT) and SOD2 (SEQ ID NO: 2, 5′-GCACACTGCCCGGCTCAACATGCTG) or the respective scrambled S-oligos (Scr). SOD protein levels were assayed at 24 h. Lane 1, control; lane 2, 10 μM each of the scrambled S-oligos; lane 3, 10 μM each of anti-SOD1 and anti-SOD2 S-oligos. 2-ME was added at 24 h and incubated for another 48-72 h followed by MTT assay. E) Effect of anti-SOD1 or random (Rd) S-oligo (10 μM) on the survival of 2-ME-treated H1299 cells (MTT, 72 h). F) Effect of antioxidants on 2-ME-induced apoptosis. Lanes 1 and 5, control HL-60 cells; lanes 2-4, cells treated with 1 μM 2-ME plus 0, 0.03, and 0.1 mM ambroxol; lanes 6-8, cells treated with 1 μM 2-ME plus 0, 3, and 5 mM N-acetylcysteine.

FIGS. 22A and 22B:

FIG. 22A: Accumulation of 2-ME in leukemia cells and normal lymphocytes (see Methods).

FIG. 22B: HPLC analysis of extracts from cells incubated with [3H]2-ME (0.5 μCi/ml, 5 h). The top two panels are chromatograms of [³H]2-ME standard monitored by UV and liquid scintillation counting, respectively. The lower three panels show the profiles of radioactivity in extracts of ML-1, HL-60, and CLL cells, respectively.

FIG. 23: Biochemical basis of combination strategies: effect of rotenone on redox status and the activity of 2-methoxyestradiol.

FIG. 24: Effect of 2-methoxyestradiol and rotenone on intracellular superoxide contents in HL-60 cells.

FIG. 25A and FIG. 25B: Synergistic activity of rotenone and 2-methoxyestradiol in HL-60 cells.

FIG. 26: The synergistic activity of rotenone and 2-methoxyestradiol is cell cycle-independent.

FIG. 27: Activation of apoptotic cascade by 2-methoxyestradiol and rotenone.

FIG. 28: Effect of rotenone and 2-methoxyestradiol on cellular ribonucleotide pools.

FIG. 29: Caspase-3 activation by oxidized and reduced forms of cytochrome c in a cell free system.

FIG. 30: Induction of DNA fragmentation by oxidized and reduced forms of cytochrome c in isolated nuclei.

FIG. 31: Effect of 2-methoxyestradiol and rotenone on colony formation in human lung cancer cells (H1299 cells).

FIG. 32A, FIG. 32B and FIG. 32C: Suppression of the cytotoxic effect of 2-methoxyestradiol by an antioxidant N-acetylcysteine in primary human chronic lymphocytic leukemia (CLL) cells.

FIG. 33: Biochemical basis for combination of ionizing radiation (IR) and 2-methoxyestradiol (2-ME).

FIG. 34A and FIG. 34B: Synergistic activity of 2-methoxyestradiol (2-ME) and ionizing radiation in human lung cancer H1299 cells.

FIG. 35: Effect of 2-methoxyestradiol on the survival of Lymphocytes from 5 healthy Donors (MTT, 72 h).

FIG. 36: Effect of 2-methoxyestradiol on the survival of Lymphocytes from 3 healthy Donors (MTT, 96 h).

FIG. 37: Effect of 2-methoxyestradiol on the survival of Leukemia Cells (MTT, 72 h).

FIG. 38: Inhibition of SOD by 2-methoxyestradiol in vitro.

FIG. 39: Effect of 2-methoxyestradiol and arsenate combination on cell survival in 2-ME-sensitive primary leukemia cells (CLL-376550) in vitro (MTT assay, 72 h incubation).

FIG. 40: Effect of 2-methoxyestradiol and arsenate combination on cell survival in 2-ME-sensitive primary leukemia cells (CLL-257266) in vitro (MTT assay, 72 h drug incubation).

FIG. 41: Effect of 2-methoxyestradiol and arsenate combination on cell survival in 2-ME-resistant primary leukemia cells (CLL-266050) in vitro (MTT assay, 72 h drug incubation).

FIG. 42: Effect of 2-methoxyestradiol and arsenate combination on cell survival in 2-ME-resistant primary leukemia cells (CLL-384854) in vitro (MTT assay, 72 h drug incubation).

FIG. 43: Combination of 2-methoxyestradiol of all-trans retinoic acid caused substantial increase of cellular superoxide content (flow cytometry analysis), and resulted in a synergistic activity against leukemia cells from a CLL patient (MTT assay).

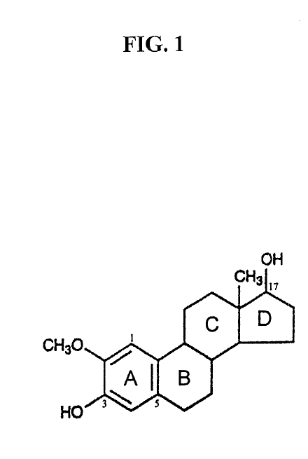

### DESCRIPTION OF ILLUSTRATIVE EMBODIMENTS

The instant invention, provides a therapeutic approach wherein a compound or compounds that inhibits SOD activity is administered in combination with an agent or agents that increases intracellular reactive oxygen intermediate accumulation. The active O₂⁻ production and low SOD activity in cancer cells renders malignant cells highly dependent on SOD for survival and sensitive to inhibition of SOD. Inhibition of SOD causes an accumulation of cellular O₂⁻ and leads to free radical-mediated damage to mitochondrial membranes, the release of cytochrome c from mitochondria, and apoptosis of the cancer cells. The inventors have determined that combining the SOD inhibiting activity of 2-methoxyestradiol with an agent that increases intracellular O₂⁻ results in a markedly enhanced killing of tumor cells. The instant invention provides compositions and methods for enhancing the antineoplastic/tumoricidal properties of an SOD inhibitor by concomitantly increasing intracellular oxygen radical concentrations with the administration of an additional agent that increases reactive oxygen intermediates within the targeted cancer cell.

A number of inhibitors of SOD activity have been identified. Unfortunately, the majority are either highly toxic or otherwise unsuitable for administration in a therapeutic regimen. 2-methoxyestradiol, a human steroid metabolite, is an inhibitor of superoxide dismutase, including cytosolic SOD1 (CuZn-SOD) and mitochondrial SOD2 (Mn-SOD), that is suitable for pharmaceutical use. Treatment of cancer cells with 2-methoxyestradiol causes an oxidative stress in the cells and triggers apoptosis in the cancer cells, with the most prominent effect to date observed in human leukemia cells. The effect is apparently specific to neoplastic cells, as no apparent cytotoxic effect is observed in normal human lymphocytes from healthy donors incubated with 2-methoxyestradiol, suggesting that certain neoplastic cells may depend more upon SOD for survival than normal cells. A potential added benefit of the administration of 2-methoxyestradiol, particularly in the context of leukemia, is that because SOD is an inhibitor of erythroid progenitor cell cycling, 2-methoxyestradiol may potentially stimulate the production of red blood cells and thus alleviate the anemic conditions often associated with leukemia.

Chemical compounds and physical modalities that enhance the generation of free radicals by-products, especially oxygen radicals, are well known in the art. While mechanisms of activity differ among the various compounds, they all facilitate the intracellular accumulation of reactive oxygen intermediates that are ultimately cytotoxic. Beyond a certain threshold concentration, reactive oxygen species elicit the onset of apoptosis by the induction of mitochondrial membrane permeability transition and release of cytochrome c (Lee, et al., 2000).

The instant invention demonstrates an enhanced killing of cells previously known to be susceptible to 2-methoxyestradiol treatment by increasing intracellular O₂⁻ in the target cells. The instant invention further contemplates that these methods and compositions will be effective against cell types previously deemed to be resistant to 2-methoxyestradiol therapy. The combination of the SOD inhibitor, 2-methoxyestradiol, with an agent that effectively increases intracellular O₂⁻ is contemplated to be effective in overcoming the ability of cells to specifically resist either composition delivered independently.

A. 2-methoxyestradiol

2-Methoxyestradiol (1,3,5(10)-estratrien-2,3,17b-triol 2 methylether (C₁₉H₇O₃)) is a multicyclic estradiol derivative with a molecular weight of 302.42. The compound has been previously shown to inhibit the formation of new blood vessels required by tumors and also to directly inhibit the growth of tumor cells. 2-methoxyestradiol is commercially available (Research Plus, Bayonne, N.J.) or may be formulated as described below.

Methods for the synthesis of estradiol and estradiol derivatives are well known in the art; see, for example Eder, (1979), and Oppolzer and Roberts, (1980). Further, methods for constructing seven membered rings in multi-cyclic compounds are well established (Nakamuru, et al., 1962, Sunagawa, et al., 1961, Van Tamelen, et al., 1961, Evans, et al., 1981). The chemical synthesis of estradiol may be readily modified to include 7-membered rings by making appropriate changes to the starting materials, so that the process of ring closure results in seven-membered rings. Known chemical methods facilitate the modification of estradiol or estradiol derivatives to include the appropriate chemical side groups according to the invention (The Merck Index, 1989), pp. 583-584).

B. Inducers of Reactive Oxygen Intermediates

1. Rotenone

Rotenone, 2R-2α,6α,12aα)-1-2,12,12a-tetra-hydro-8,9-dimethoxy-2-(1-meth-ylethenyl)-[1] benzopyrano-[3,4-b] furo[2,3-h] [1]benzyopyran-6(6aH)-one (C₂₃H₂₂O₆)) is a multicyclic, naturally occurring substance found in the roots and stems of several tropical plants. Extracts of these plants have been used for centuries as potent pesticides and insecticides. Rotenone works by inhibiting mitochondrial electron transport, resulting in an inability of the cell to use oxygen in the release of energy during normal body processes. In effect, the cells suffocate due to lack of oxidative phosphorylation. The cytotoxic activity of rotenone is attributed to its inhibition of NADH:ubiquinone oxidoreductase activity, while its potential cancer chemopreventive effect of has been associated with inhibition of phorbol ester-induced ornithine decarboxylase (ODC) activity. Further study suggests that the molecular features of rotenone essential for inhibiting NADH:ubiquinone oxidoreductase are similar to those for blocking ODC induction, with the IC₅₀ values in the range of 0.8-4 nM. It is proposed that inhibition of NADH:ubiquinone oxidoreductase activity lowers the level of induced ODC activity, leading to the antiproliferative effect and anticancer action (Fang et at, 1998). In the context of the instant invention, it is contemplated that inhibition of the mitochondrial electron transport by rotenone will cause an increase of intracellular superoxide radicals and, when combined with 2-methoxyestradiol to inhibit SOD, will result in a severe superoxide stress and produce synergistic anticancer activity.

2. Tumor Necrosis factor-alpha

Tumor necrosis factor-alpha (TNF-α) is a naturally occurring cytokine involved in a variety of biological processes including inflammation, cancer cachexia, and regulation of cell growth and apoptosis. This molecule is known to induce production of reactive oxygen species and to cause apoptosis in cells. It is demonstrated that the TNF-alpha-induced apoptosis through production of superoxide anion, which function as the crucial mediator for the TNF-alpha-initiated apoptotic pathway (Moreno-Manzano et al, 2000). TNF-alpha has been used in clinical trials in a variety of human cancer patients. For example, combination of TNF-alpha and interleukin-2 significantly improved the response rate of patients with metastatic renal cell carcinoma compared to treatment with either agent alone (Bukowski, 2000). TNF-alpha has also been used in combination with melphalan and hyperthermia in treating patients with malignant melanoma or breast cancer, with induction of complete remissions (Robins, 1999). TNF-alpha is administered intravenously, with a dose range of 50-100 micrograms/m². The dose-limiting toxicity include myelosuppression and thrombocytopenia. It is suggested that high doses of TNF-alpha may be administered by isolated limb perfusion to treat unresectable sarcoma and melanoma (Fraser et al., 1999). In the context of the instant invention, TNF-alpha could be combined with 2-methoxyestradiol to cause enhanced accumulation of intracellular superoxide radicals and thus produce synergistic anticancer activity.

3. Bleomycin Bleomycin A₂, Ni [3-(dimethylsulfonio)propyl]bleomycinamide (C₅₅H₈₄N₁₇O₂₁S₃), is a glycopeptide member of the bleomycin peptide family isolated from Streptomyces verticillus. Bleomycins are known to cause strand scission of DNA as well as possessing oxygen transferase activity. Strand scission occurs because of free radical generation from the interaction of bleomycin, iron, and oxygen. The compounds are employed as antimicrobial and antineoplastic agents.

Bleomycin is poorly absorbed across the GI tract and must be administered parenterally. Bleomycin may be administered intramuscularly, intravenously, subcutaneously or intrapleurally. The general dosage employed in treatment regimens is 0.25 to 0.5 units/kg (10-20 units/m²) intramuscularly, intravenously or subcutaneously, delivered at weekly or bi-weekly intervals. Pulmonary toxicity of the drug is dose dependent with a striking increase in toxicity above 400 units.

4. Cisplatin

Cisplatin, cis-Diamminedichloroplatinum (Cl₂H₆N₂Pt), is a heavy metal complex containing a central atom of platinum surrounded by two chloride atoms and two ammonia molecules in the cis position. Cisplatin cytotoxicity is based upon a number of alternate effects including DNA binding, mitochondrial damage, decreased ATPase activity, and altered cellular transport mechanisms. After entering cells by diffusion, cisplatin becomes chemically active due to the loss of its chloride ions by hydrolysis. Free radical intermediates are produced as the consequence of the chemical reactions. The drug's efficacy is based upon its stereochemistry, as the trans isomer is not cytotoxic. Cisplatin is a non-cell cycle specific, bifunctional, alkylating agent with activity against both solid tumors and lymphoma. Cisplatin is currently the treatment of choice in many testicular, bladder, gastric, head and neck, non-small cell lung, ovarian, and small cell lung cancers.

Cisplatin is administered primarily through IV infusion although intra-arterial delivery is also possible. Dosage of cisplatin varies from 20 mg/m² to over 100 mg/m² depending upon the nature of the neoplasia treated. Renal and hematologic function must be assessed prior to the administration of cisplatin. Dosage in excess of 100 mg/m² must be closely monitored due to the risk of overdosage and platinum toxicity. To decrease the incidence and severity of nephrotoxicity, hydration consisting of NS 250 ml/hr for 2-4 hours prior to and post cisplatin, and maintenance of urine output of 100-150 ml/hr for 24 hours following cisplatin are recommended. In addition, premedication with antiemetics including a serotonin antagonist and corticosteroid prevents the severe nausea and vomiting associated with the drug.

5. Anthracycline Derivatives

The anthracycline antibiotics were initially derived from the fungus Streptomyces peuceitius var. caesius. Synthetic members of this family have also been produced. Anthracyclines achieve their cytotoxic effect by several mechanisms, including: inhibition of topoisomerase II; intercalation between DNA strands, thereby interfering with DNA and RNA synthesis; production of free radicals that react with and damage intracellular proteins and nucleic acids; chelation of divalent cations; and reaction with cell membranes. The wide range of potential sites of action may account for the broad efficacy as well as the toxicity of the anthracyclines (Young et al., 1985).

The anthracycline antibiotics have tetracycline ring structures with an unusual sugar, daunosamine, attached by glycosidic linkage. Cytotoxic agents of this class all have quinone and hydroquinone moieties on adjacent rings that permit them to function as electron-accepting and donating agents.

a. Doxorubicin

Doxorubicin hydrochloride, 5,12-Naphthacenedione, (8s-cis)-10-[(3-amino-2,3,6-trideoxy-α-L-lyxo-hexopyranosyl)oxy]-7,8,9,10-tetrahydro-6,8,1-trihydroxy-8-(hydroxyacetyl)-1-methoxy-hydrochloride (hydroxydaunorubicin hydrochloride, Adriamycin) is used in a wide antineoplastic spectrum. Doxorubicin exerts its cytotoxic effect on tumor cells mainly by two mechanisms: (a) generation of reactive oxygen species (ROS); and (b) inhibition of topoisomerase II.

Doxorubicin is absorbed poorly and must be administered intravenously. The pharmacokinetics are multicompartmental. Distribution phases have half-lives of 12 min and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Children, 30 mg/m² on each of 3 consecutive days, repeated every 4 wk. Prescribing limits are as with adults.

b. Daunorubicin

Daunorubicin hydrochloride, 5,12-Naphthacenedione, (8S-cis)-8-acetyl-10-[(3-amino-2,3,6-trideoxy-α-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin undergoes reduction to form oxygen free radical intermediates. In the presence of oxygen and metal catalysts such as Fe²⁺, daunorubicin undergoes reduction to the semiquinone radical. In the presence of oxygen, the semiquinone radical can form a superperoxide that in the presence of hydrogen peroxide forms hydroxyl radicals.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 min and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr 45 mg/m2/day (30 mg/m2 for patients older than 60 yr) for 1, 2 or 3 days every 3 or 4 wk or 0.8 mg/kg/day for 3 to 6 days every 3 or 4 wk; no more than 550 mg/m2 should be given in a lifetime, except only 450 mg/m² ir there has been chest irradiation; children, 25 mg/m2 once a week unless the age is less than 2 yr or the body surface less than 0.5 m, in which case the weight-based adult schedule is used. It is available in injectable dosage forms (base equivalent) 20 mg (as the base equivalent to 21.4 mg of the hydrochloride).

C. Epirubicin

Epirubicin, (8 S-cis)-10-[(3-amino-2,3,6,-trideoxy-α-L-arabino-hexopyranosyl) oxyl]-7,8,9,10-tetrahydro-6,8,11-trihydroxy-8-(hydroxyacetyl)-1-methoxy-5,12-naptha-cenedione (C₂₇H₂₉NO₁₁), is an anthracycline derived chemotherapeutic agent which is the 4′-epimer of doxorubicin and a semi-synthetic derivative of daunorubicin. Epirubicin undergoes one-electron reduction to form oxygen free radical intermediates. In the presence of oxygen and metal catalysts such as Fe²⁺, epirubicin is reduced to the semiquinone radical. In the presence of oxygen, the semiquinone radical can form a superperoxide that in the presence of hydrogen peroxide forms a hydroxyl radical.

Epirubcin is administered intravenously by infusion over 30-60 minutes rather than by direct injection. The drug is extremely irritating to tissues and should thus not be administered intramuscularly or subcutaneously.

6. Arsenic Compounds

Arsenic agents have been used in ancient Chinese medicine for several diseases. One of these agents, arsenic trioxide, (As₂O₃), has recently been reported to be an effective agent in therapy for relapse or refractory acute promyelocytic leukemia. Data also suggests the therapeutic use of arsenic trioxide for other hematologic cancers as well (Soignet et al., 1999a; Wiemik et al., 1999, Geissler et al., 1999; Rousselot et al., 1999). Organic arsinals, such as melarsoprol (Soignet et al., 1999b) and an arsenic pyrimidine compound (U.S. Pat. No. 6,191,123) may also have therapeutic implications.

7. Retinoic Acid Derivatives

Retinoic acids such as all-trans retinoic acid (ATRA) or 9-cis retinoic acid are anticancer agent used in the clinical treatment of cancer. It is a known regulator of cellular proliferation and differentiation, and a known inhibitor of tumor promotion. ATRA has been found to be especially useful in hematological malignancies such as acute promyelocytic leukemia by causing differentiation and apoptosis in immature malignant promyelocytes. Doses of ATRA are normally about 45 mg/m2 per day.

8. Physical Agents that Generate Free Radicals

a. Heat (hyperthermia)

Hypothermia is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radio-frequency electrodes. It is know that hyperthermia causes an increased flux of free radicals in cells (Flanagan, 1998).

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

b. Ultraviolet Rays

Ultraviolet (UV) rays are invisible radiation with wavelength of less than 400 nm. Ultraviolet rays are subdivided into three regions, UVA (340-400 nm), UVB (290-320 run), and UVC (200-290 nm). It is well known in the art that UV has various effects on biological system, depending on the wavelength and the intensity of the radiation. For example, ultraviolet at certain range of wavelength causes damage to cellular DNA by formation of pyrimidine dimers. The rate of induction of pyrimidine dimers is maximal at 254 nm (Freeman et al, 1990). The UV-induced DNA damage is believed to be a major mechanism responsible for the mutagenic and carcinogenic effects of this type of radiation. At a sufficient radiation intensity, UV causes cell death by induction of apoptosis. It is also known in the art that UV rays, especially at the wavelength of 280-400 nm, cause production of reactive oxygen species. These free radicals subsequently damage cells and cause apoptosis (Paretzoglou et al, 1998; Nishi et al, 1991; Nishigaki et al, 1999). In the context of the instant invention, it is contemplated that the use of 2-methoxyestradiol to inhibit SOD in combination with a local ultraviolet radiation at the tumor site, especially the tumor of skin, may cause a preferential accumulation of free radicals in the cancer cells, and thus enhance the potency and selectivity of cancer therapy.

C. X-rays and Gamma-rays

X-rays (roentgen rays) and Gamma-rays are two major types of electromagnetic radiation wildly used in the medical field. In atomic terms, X-rays are produced extranuclearly while gamma rays are produced intranuclearly. The generation of electromagnetic radiation and its used in medicine are well known in the art. When electromagnetic radiation is applied to a biological system, the energy of the radiation causes damage to cells by two possible mechanisms. (1) The direct effect on important target molecules such as DNA, which can be structurally damages and modified. (2) production of free radicals by interaction of the energy rays with water inside the cells or in the tissue matrix. The free radicals then interact with important biological molecules such as DNA and protein, and cause detrimental effects. In the context of the instant invention, it is contemplated that the use of 2-methoxyestradiol to inhibit SOD in combination with a local application of X-rays or gamma radiation at the tumor site may cause a preferential accumulation of free radicals in the cancer tissue, and thus enhance the potency and selectivity of cancer therapy.

C. Combination Therapy

It is contemplated that the methods and compositions of the instant invention will demonstrate effective tumoricidal properties, nevertheless, it may be desirable, in certain circumstances to combine the claimed therapeutic approach with other cancer treatments. Thus, the claimed compositions may be combined with other agents effective in the treatment of hyperproliferative disease, such as other anti-cancer agents, or with surgery. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell.

1. Chemotherapy

Cancer therapies include a variety of combination therapies with both chemical and radiation based treatments. Combination chemotherapies include, for example, carboplatin, procarbazine, mechlorethamine, cyclophosphamide, camptothecin, ifosfamide, melphalan, chlorambucil, busulfan, nitrosurea, dactinomycin, plicomycin, mitomycin, etoposide (VP16), tamoxifen, raloxifene, estrogen receptor binding agents, taxol, gemcitabine, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. In the context of the present invention, it is contemplated that the disclosed methods and compositions could be used in conjunction with one or more of the foregoing chemotherapeutic agents. The combination of chemotherapy with biological therapy is known as biochemotherapy.

2. Gene Therapy

Tumor cell resistance to chemotherapy and radiotherapy agents represents a major problem in clinical oncology. One goal of current cancer research is to find ways to improve the efficacy of chemotherapy, as for example, claimed in the instant invention, by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that gene therapy could be used similarly in conjunction with the disclosed methods and compositions.

Various combinations of therapies may be employed, the claimed methods and compositions are “A” and the secondary agent, such as radio- or chemotherapy, gene therapy or surgery, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary. It also is contemplated that various standard therapies, as well as surgical intervention, may be applied in combination with the described therapy.

a. Inducers of Cellular Proliferation

The proteins that induce cellular proliferation further fall into various categories dependent on function. The commonality of all of these proteins is their ability to regulate cellular proliferation and thus affect cellular metabolisms. For example, the gene c-myc is known to stimulate cell proliferating activity and cause an increase in production of free radical intermediates. In one embodiment of the present invention, it is contemplated that an ectopic overexpression of c-myc by gene transfer could be used in conjunction with the disclosed methods and compositions to kill cancer cells. As a second example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor genes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16^{INK4} belongs to a newly described class of CDK-inhibitory proteins that also includes p₁₆^{B}, p19, p21^{WAF1}, and p₂₇^{KIP1}. The p16^{INK4} gene maps to 9p21, a chromosome region frequently deleted in many tumor types. Homozygous deletions and mutations of the p16^{INK4} gene are frequent in human tumor cell lines. Other genes that may be employed according to the present invention include Rb, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fins, trk, ret, gsp, hst, abl, E1A, p300, genes involved in angiogenesis (e.g., VEGF, FGF, thrombospondin, BAI-1, GDAIF, or their receptors) and MCC.

C. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al, 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, BCl_{S}, MCl-1, Al, Bfl-1) or counteract Bcl-2 function and promote cell death (e.g., Bax, Bak, Bik, Bim, Bid, Bad, Harakiri).

3. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

4. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy may be useful as part of a combined therapy. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

a. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al., (1988). The development of human monoclonal antibodies is described in further detail elsewhere in the specification.

b. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

C. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

D. Pharmaceutical Compositions

1. Pharmaceutically Acceptable Carriers

Aqueous compositions of the present invention comprise an effective amount of 2-methoxyestradiol dissolved and/or dispersed in a pharmaceutically acceptable carrier and/or aqueous medium and an effective amount of a compound that increases intracellular O₂⁻ concentration dissolved and/or dispersed in a pharmaceutically acceptable carrier and/or aqueous medium. The phrases “pharmaceutically and/or pharmacologically acceptable” refer to molecular entities and/or compositions that do not produce an adverse, allergic and/or other untoward reaction when administered to an animal, and specifically to humans, as appropriate.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For administration to humans, preparations should meet sterility, pyrogenicity, general safety and/or purity standards as required by FDA Office of Biologics standards.

The 2-methoxyestradiol and a compound that increases intracellular O₂⁻ concentration should be extensively purified to remove undesired small molecular weight molecules and/or lyophilized for more ready formulation into a desired vehicle, where appropriate. The active compounds will then generally be formulated for oral administration, or for parenteral administration, e.g., formulated for injection via the intravenous, intramuscular, sub-cutaneous, intralesional, and/or even intraperitoneal routes. The preparation of an aqueous composition that contains a 2-methoxyestradiol agent and/or the compound that increases intracellular O₂⁻ concentration as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

The 2-methoxyestradiol and a compound that increases intracellular O₂⁻ concentration can be formulated into a composition in a neutral and/or salt form. Pharmaceutically acceptable salts, include the acid addition salts (formed with the free amino groups of the protein) and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

2-methoxyestradiol and a compound that increases intracellular O₂⁻ concentration may be formulated within a therapeutic mixture to comprise about 0.0001 to 1.0 milligrams, and/or about 0.001 to 0.1 milligrams, and/or about 0.1 to 1.0 and/or even about 10 milligrams per dose and/or so. Multiple doses can also be administered.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Various commercial nasal preparations are known and/or include, for example, antibiotics and/or antihistamines and/or are used for asthma prophylaxis.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

2. Liposomes and/or Nanocapsules

In certain embodiments, the use of liposomes and/or nanoparticles is contemplated for the introduction of 2-methoxyestradiol and the compound the increases intracellular O₂⁻ concentration into host cells. The formation and/or use of liposomes is generally known to those of skill in the art, and/or is also described below.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and/or such particles may be are easily made.

Liposomes are formed from phospholipids that are dispersed in an aqueous medium and/or spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The following information may also be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs.

Liposomes interact with cells via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

E. Kits

Therapeutic kits of the present invention are kits comprising 2-methoxyestradiol and a compound that increases intracellular O₂⁻ concentration. Such kits will generally contain, in suitable container means, and a pharmaceutically acceptable formulation of 2-methoxyestradiol a compound that increases intracellular O₂⁻. The kit may have a single container means, and/or it may have distinct container means for each compound.

When the components of the kit are provided in one and/or more liquid solutions, the liquid solution is an aqueous solution, with a sterile aqueous solution being particularly preferred. 2-methoxyestradiol compositions and compositions of a compound that increases intracellular O₂⁻ concentration may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The components of the kit may be provided as solid tablets or capsules containing 2-methoxyestradiol and tablets or capsules containing a compound that increases intracellular O₂⁻ concentration for oral administration, either simultaneously or sequentially with the tablets/capsules containing each component.

The container means will generally include at least one vial, test tube, flask, bottle, syringe and/or other container means, into which the 2-methoxyestradiol and a compound that increases intracellular O₂⁻ concentration are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the 2-methoxyestradiol composition and a composition with a compound that increases intracellular O₂⁻ concentration within the body of an animal. Such an instrument may be a syringe, pipette, forceps, and/or any such medically approved delivery vehicle.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Methods

Assay of SOD Activity

A spectrophotometric assay was used to determine the effect of 2-methoxyestradiol (2-ME) on SOD activity in vitro with purified enzymes (bovine CuZnSOD from Boehringer Mannheim; human CuZnSOD and E. coli MnSOD from Sigma). The reactions contained 2.5 ml of 50 mM Na₂CO₃, 0.1 ml of 3 mM xanthine, 0.1 ml of 3 mM EDTA, and 0.1 ml of 0.8 mM XTT (3′-(1-[phenylamino-carbonyl]3,4-tetrazolium)-bis(4-methoxy-6-nitro)benzene-sulfonic acid hydrate), and the indicated concentrations of SOD and 2-ME. Xanthine oxidase (0.1 ml, 64 mU/ml) was added to start the reaction. After incubation at 24° C. for 30 min, the absorbance at 470 nm was measured. The relative activity (RA) of SOD was calculated by the following formula:

RA=[1−(c−b)/(a−b)]×100%

where a is the absorbance of reaction without SOD, b is the absorbance of reaction with SOD but without 2-ME, c is the absorbance of reaction with SOD in the presence of 2-ME. All values were subtracted by the background reading of the blank. In separate experiments using nitro blue tetrazolium or cytochrome c as substrates for SOD assay, 2-ME and DMSO (as a solvent for 2-ME) interfered with the calorimetric reaction. These artifacts preclude the use of NBT or cytochrome c as the substrates for measuring the effect of 2-ME on SOD activity. Neither 2-ME nor DMSO affect the calorimetric reaction of XTT.

Quantitation of Cellular O₂⁻ and H₂O₂ Production

The method using hydroethidine to detect O₂⁻ in tissue sections was adapted for quantitation of intracellular O₂⁻ by flow cytometry analysis. This assay is based on the unique chemical properties of hydroethidine, a weak blue fluorescent dye which is selectively converted by O₂⁻ to ethidium with a bright red fluorescence. The control and drug-treated cells (1.5×10⁶/sample) were incubated with hydroethidine (20 ng/ml) for 60 min, washed twice with 3 ml PBS, resuspended in 2 ml PBS, and then analyzed within 1 h by flow cytometry, using the red laser channel. A microassay was used to measure the effect of 2-ME on H₂O₂ generation in cell culture. Exponentially growing cells were harvested, suspended at the density of 10⁷/ml, and incubated with 2-ME in a modified KRPG solution containing 145 mM NaCl, 5.7 mM Na₂HPO₄ (pH 7.35), 4.86 mM KCl, 0.54 mM CaCl₂, 1.22 mM MgSO₄, horseradish peroxidase (type II, 1 unit/ml), and 50 μM N-acetyl-3,7-dihydroxyphenoxazine (A6550). The horseradish peroxidase converted A6550 (non-fluorescent) to a highly fluorescent product in the presence of H₂O₂. The intensity of the fluorescence was linearly proportional to H₂O₂ as long as the ratio of A6550:H₂O₂ was greater than 5 in the reaction mixtures. Samples were incubated in a 96-well plate for 2 h and then read at 590/645 nm (excitation/emission wavelengths). Various concentrations of pure H₂O₂ were used in parallel reactions to construct a standard curve.

Assay of Gene Expression by cDNA Microarray Analysis

The Atlas™ Human cDNA Expression Array I and the Atlas™ Human Cancer cDNA Expression Array (ClonTech Laboratories, Inc.) were used in this study. One microgram of mRNA isolated from the control or drug-treated cells was converted to radioactive cDNA by reverse transcription in the presence of [α-³²P]-dATP. The ³²P-labeled cDNA was then denatured and hybridized to the eDNA expression arrays according to the procedures recommended by the manufacturer. The radioactivity on the membranes was quantified by a phosphoimager. Change in gene expression after 2-ME treatment was calculated as percentage of the untreated cells, using three of the internal controls (ubiquitin, GAPDH, and the 23 kD highly basic protein) recommended by the manufacturer for normalization to ensure the comparability of the control and drug-treated samples.

RT-PCR Analysis

The expression of SOD RNA was also measured by RT-PCR. RNA (1 μg) isolated from the control or 2-ME-treated cells was first converted to cDNA by reverse transcription using SuperScript II reverse transcriptase (GibcoBRL) and the anchored oligo-dT primer set (Genosys). The cDNA was than amplified by PCR. The SOD1primers were from Genosys (forward, SEQ ID NO: 3, 5′-ACGAAGGCCGTGTGCGTGCTGAA; backward, SEQ ID NO: 4, 5′-ACCACAAGCCAAACGACTTCCAGC). The reaction was run at 94° C. (1 min)→60° C. (1 min)→72° C. (1 min) for 20 cycles, which was within the linear reaction window. GAPDH was also measured by RT-PCR from the same RNA samples and used as an internal control (GAPDH primers: forward, SEQ ID NO: 5, 5′-CCATCAATGACCCCTTCA TTGACC; backward, SEQ ID NO: 6, 5′-GAAGGCCATGCCAGTGAGCTTCC).

Assays of Cellular Accumulation and Metabolism of 2-ME

To determine cellular uptake of 2-ME, cells were incubated with 1 μM [³H]2-ME (0.5 μCi/ml) for 5 h and when washed twice with cold PBS. Radioactivity associated with the cell pellets or the culture medium was quantitated by liquid scintillation counting. 2-ME concentrations were calculated based on the specific radioactivity of [³H]2-ME, cell number, and the mean cell volume as measured by a Coulter Counter equipped with a 256-Channelyzer. HPLC was used to analyze potential cellular metabolites of 2-ME. Cells were incubated with [³H]2-ME (0.5 μCi/ml) for 5 h, washed twice with PBS, and then extracted with 50% methanol. The extracts were analyzed by HPLC equipped with a UV detector (284 nm) and an on-line liquid scintillation counter, using a μBondapak C₁₈ reversed-phase column and the following running conditions: 1 ml/min; 0-5 min, 100% buffer A (water:acetontrile:acetic acid=59:40:1); 5-17 min, a linear gradient of 0->100% buffer B (water:acetontrile:acetic acid=39:60:1); 17-30 min, 100% B.

Results

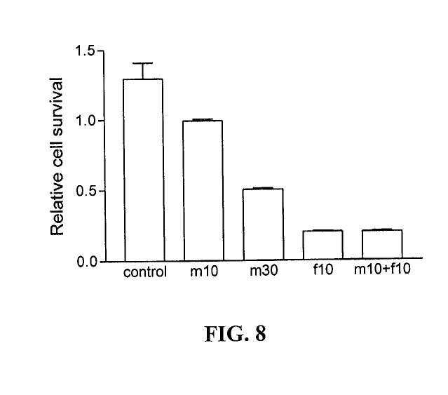

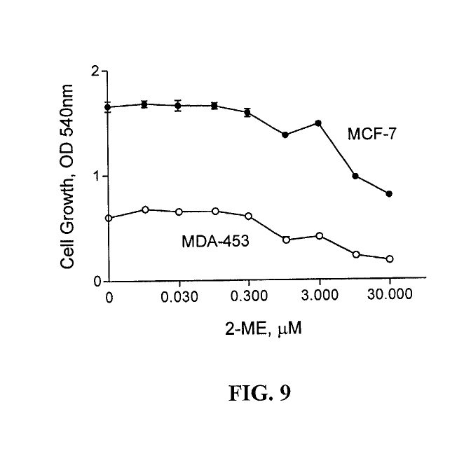

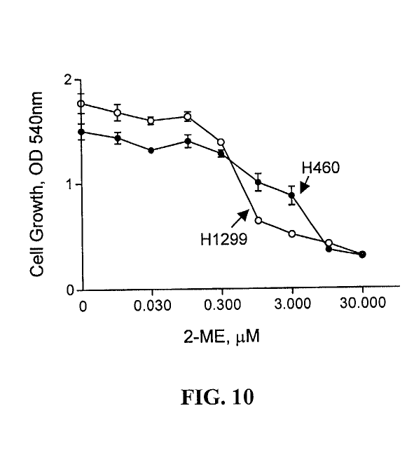

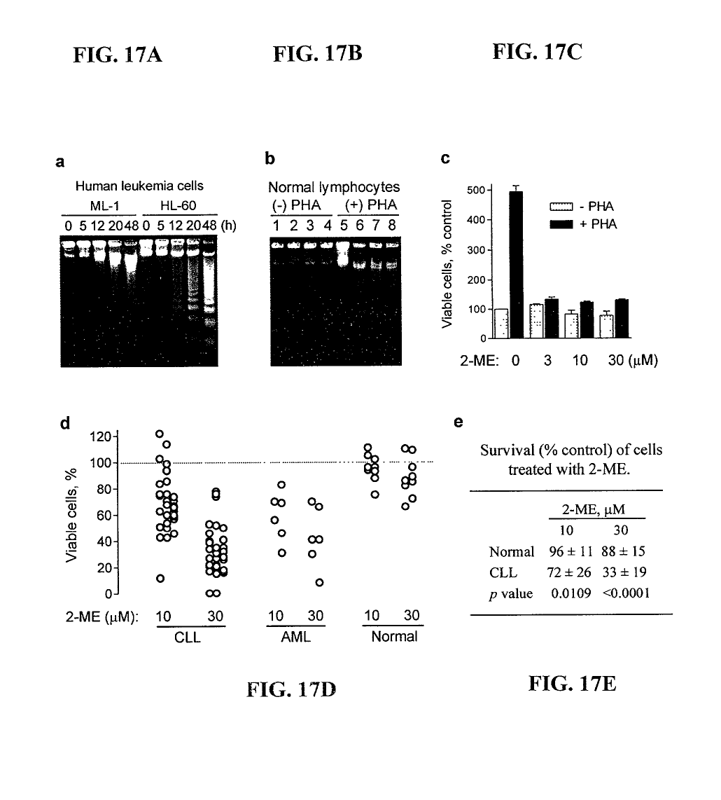

2-ME was originally employed to increase p53 protein in an attempt to enhance cellular response to other anticancer agents. Surprisingly, 2-ME itself showed a potent activity against human leukemia cells with different p53 genotypes. Typical apoptotic morphology and nucleosomal DNA fragmentation were observed in ML-1 (wt p53) and HL-60 (p53⁻) cells treated with 1 μM 2-ME (FIG. 17A). The MTT assay further demonstrated that cells with wt p53 (ML-1), mutant p53 (CEM and Raji), and no p53 (K562 and HL-60) were similarly sensitive to 2-ME (FIGS. 8-10). The IC₅₀ was less than 1 μM for all five cell lines.

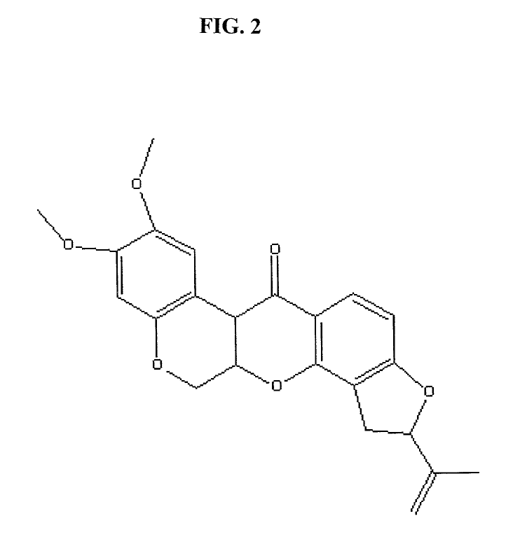

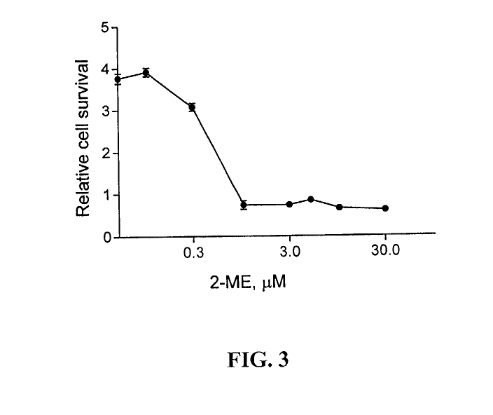

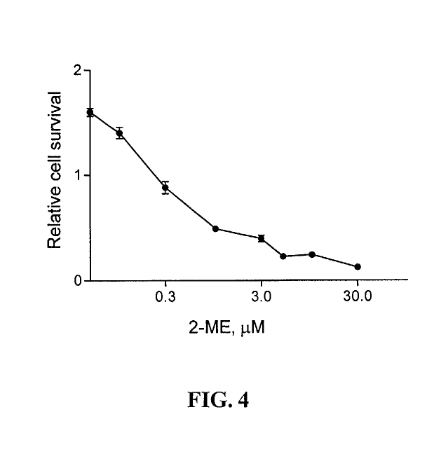

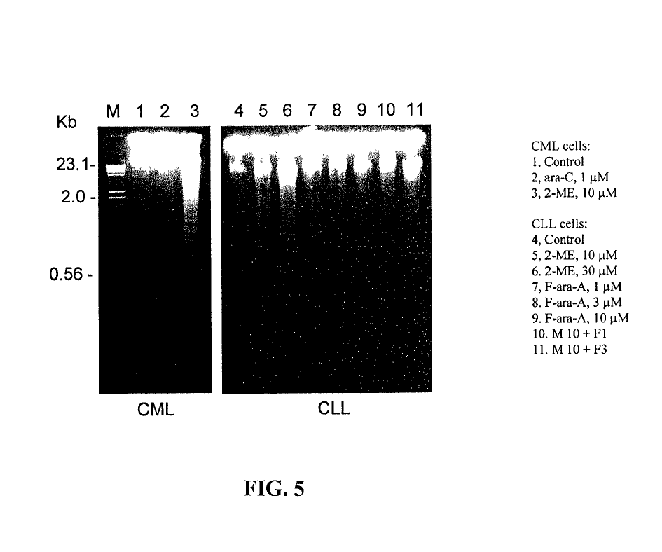

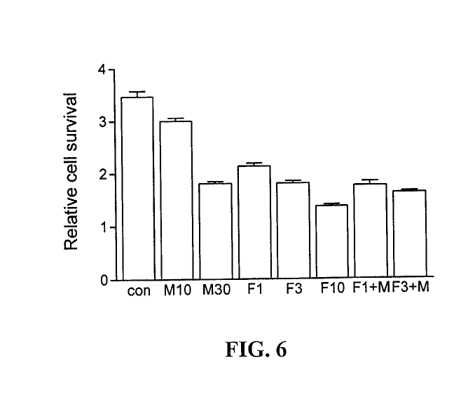

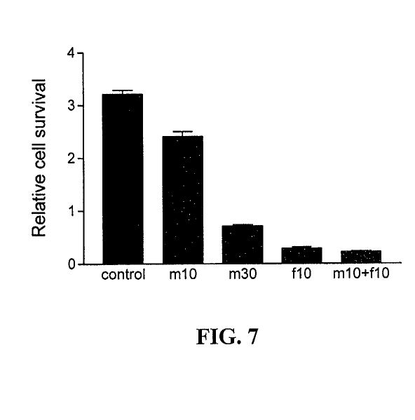

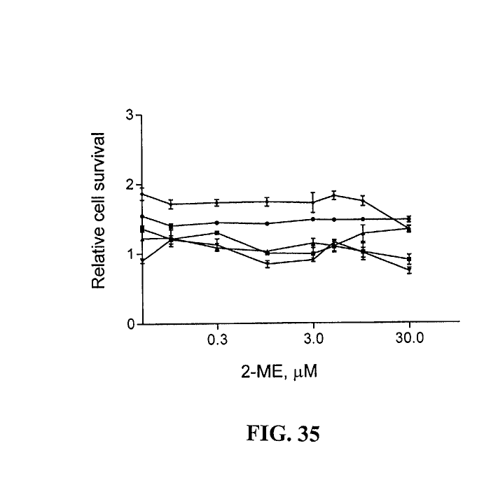

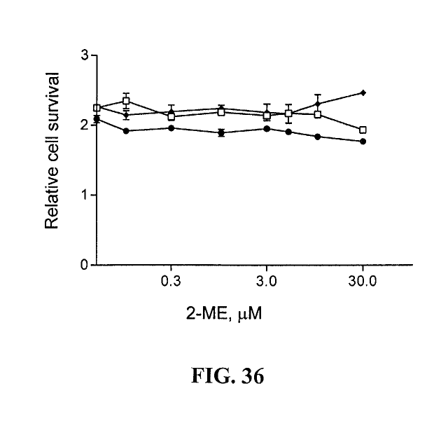

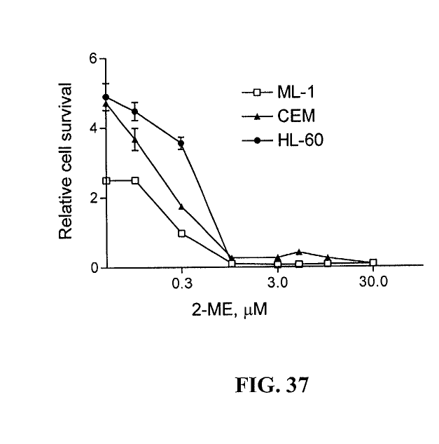

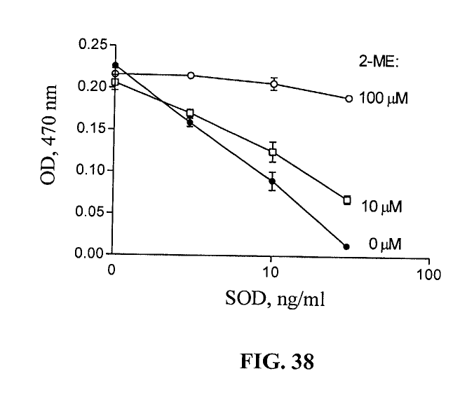

When normal lymphocytes from 8 healthy donors were incubated with 2-ME, no apparent loss of cell survival was observed (FIGS. 35-36), suggesting that 2-ME may have a selective anti-leukemia activity. Because normal lymphocytes were quiescent and might not be comparable to proliferating leukemia cells, we further used quiescent leukemia cells isolated from patients with chronic lymphocytic leukemia (CLL) for comparison with normal lymphocytes. As shown in FIGS. 6-8, 2-ME caused a substantial loss of cell survival in CLL cells. This compound also induced apoptosis in leukemia cells from a CML (chronic myelogenous leukemia) patient in relapsed blast crisis (FIG. 5e, lane 3), that were resistant to ara-C in vitro (lane 2). Evaluation of the activity of 2-ME in primary leukemia cells from a total of 31 CLL patients, 3 CML patients, 6 AML (acute myelogenous leukemia) patients, and 2 ALL (acute lymphocytic leukemia) patients revealed a substantial activity in the majority of the cases, although the degree of cytotoxicity varied among samples (FIG. 17D). Overall, 30 μM 2-ME reduced cell survival to 32.7% in CLL cells, 54.7% in CML cells, 49.6% in AML cells, and 56.5% in ALL cells. 2-ME was significantly more toxic to CLL cells than to normal lymphocytes at both 10 and 30 μM (p<0.0001, FIG. 17E).

To test effect of 2-ME in the normal lymphocytes induced to re-enter the proliferating cell cycle, lymphocytes were first stimulated with phytohemagglutinin (PHA-M) and then exposed to 2-ME. PHA (5 μg/ml, 48 h) induced significant proliferative activity, as measured by [³H]thymidine incorporation (6,903 dpm->99,875 dpm) and by flow cytometry analysis (S phase: 0%->17.1%; G2/M phases: 0%->9.5%; n—3). When the lymphocytes were incubated with 2-ME (0-30 μM) for an additional 3 days, no nucleosomal DNA fragmentation was detected (FIG. 17B, lanes 1-4). A small amount of DNA smearing was seen in all PHA-stimulated cells (lanes 5-8), suggesting some spontaneous turnover of the cells during the 5-day incubation. MTT assay revealed that 2-ME inhibited the proliferation of the PHA-treated lymphocytes, which nevertheless remained viable during the 3-day drug incubation (FIG. 17C).

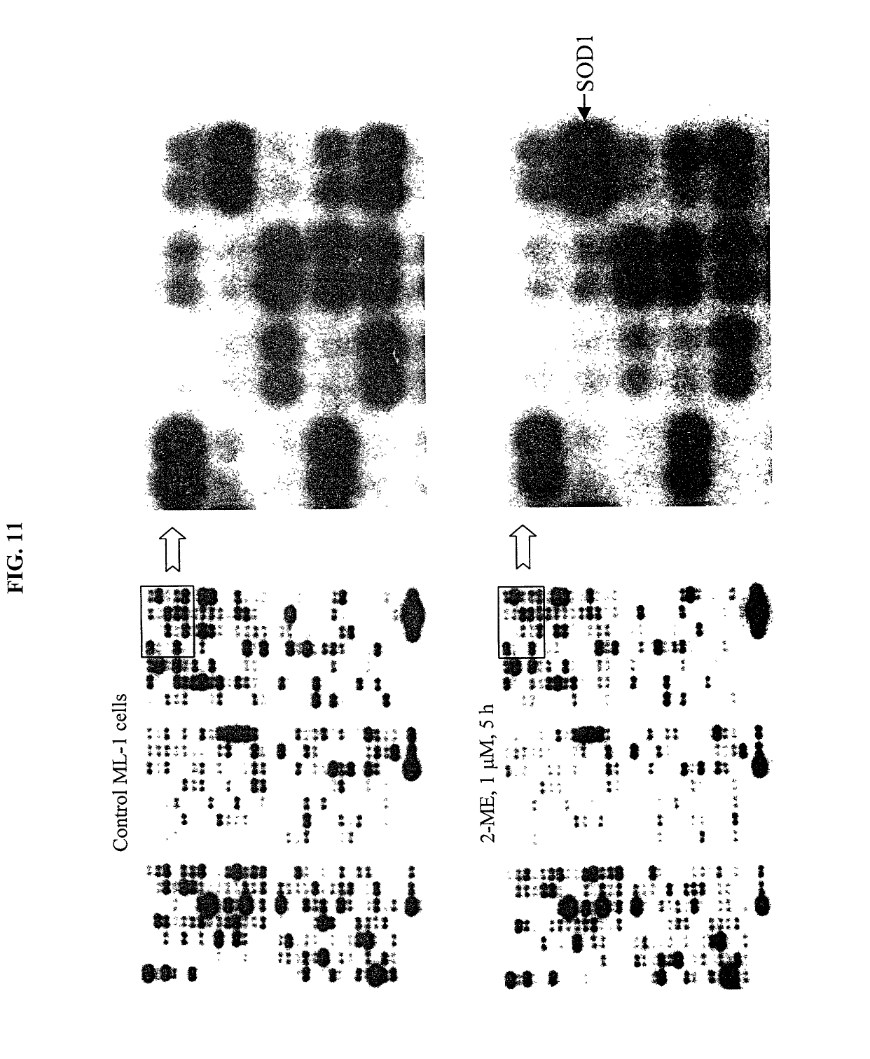

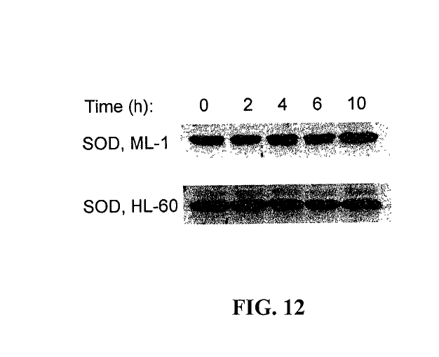

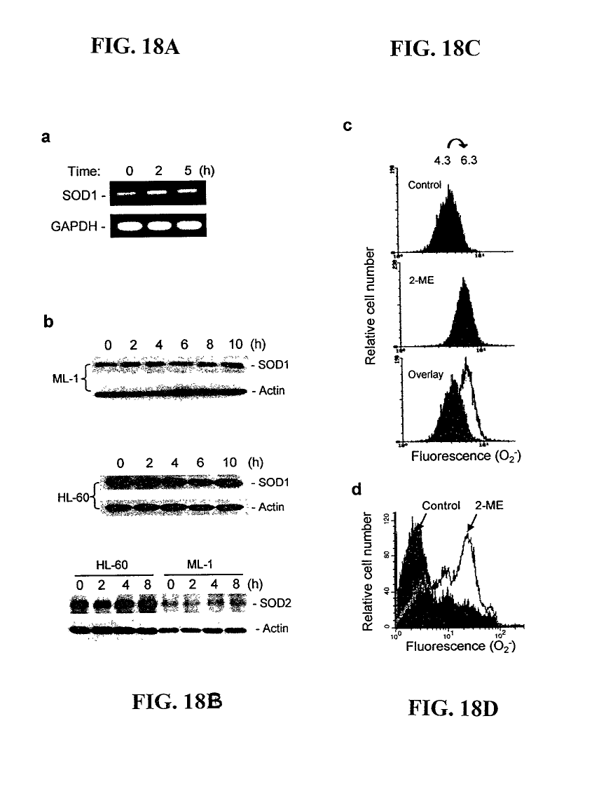

The cDNA microarray assay (Atlas Human cDNA Expression Array, ClonTech) was used to search for candidate molecules involved in the cellular responses to 2-ME. As illustrated in FIG. 11, the gene located in the second row of the last column (in doublets.) showed a significant increase of mRNA expression 5 h after 2-ME incubation. This molecule was identified as CuZnSOD (SOD1, GenBank accession code, k00065). Quantitation by phosphoimager revealed that expression of CuZnSOD in 2-ME-treated cells was 236% of the control, after the signal was normalized by three internal standards (see Methods). This increase was confirmed by a RT-PCR assay (FIG. 18A). Similar increase of CuZnSOD mRNA expression was also observed in HL-60 cells.

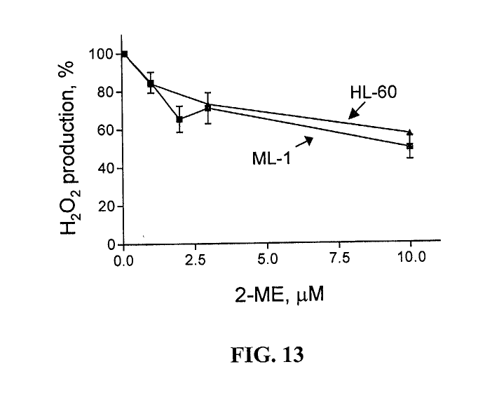

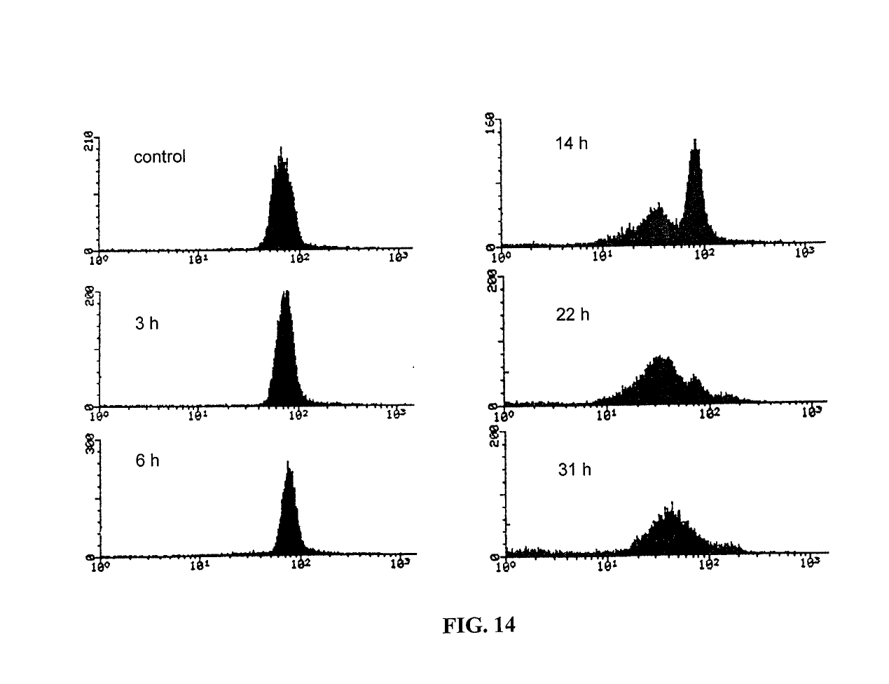

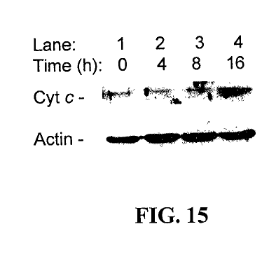

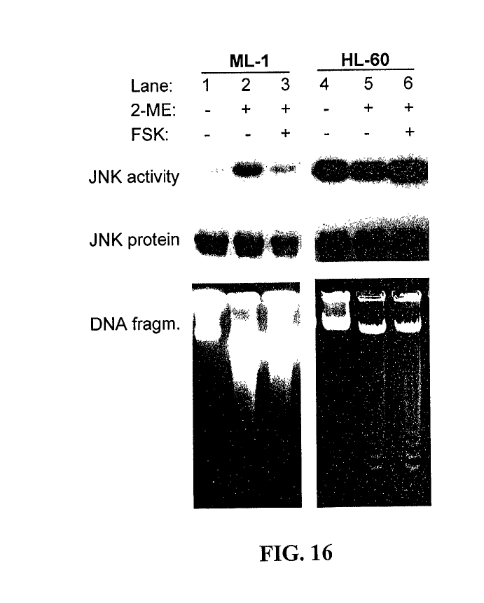

To further investigate the mechanism responsible for the increase in SOD mRNA expression, the following two possibilities were tested: (1) 2-ME might cause an increase of cellular O₂⁻ production and thus induce SOD expression in response to the free radical stress. This would lead to an increase of H₂O₂ production. (2) 2-ME might inhibit SOD enzyme activity and cause a feedback upregulation of SOD expression. This would predict a decrease in H₂O₂ generation. In fact, 2-ME caused a concentration-dependent decrease of H₂O₂ in ML-1 and HL-60 cells (FIG. 13). Immunoblot analysis showed that the reduced H₂O₂ production was not due to a loss of CuZnSOD or MnSOD protein (FIG. 18B). Rather, a moderate increase (74%) of SOD1 protein was seen in ML-1 cells at 10 h. Thus, the increase of SOD mRNA expression likely reflected a cellular response to O₂⁻ accumulation due to SOD inhibition by 2-ME. To confirm the accumulation of O₂⁻ in 2-ME-treated cells, we used hydroethidine, a compound specifically converted by O₂⁻ to highly fluorescent ethidium, to measure the cellular O₂⁻ contents by flow cytometry analysis. FIG. 17C shows that treatment of ML-1 cells with 1 μM 2-ME for 5 h caused an increase of O₂⁻ from 4.3 to 6.3 arbitrary units. Incubation of CLL cells with 30 μM 2-ME also led to a substantial O₂⁻ increase (226±37%, FIG. 18D).

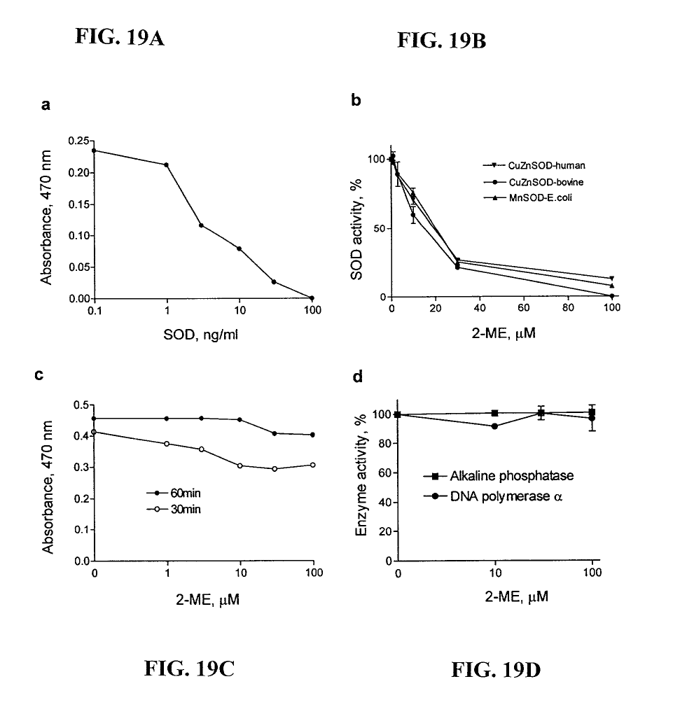

An in vitro assay for SOD activity was used to test the direct effect of 2-ME on purified CuZnSOD and MnSOD. The xanthine/xanthine oxidase system was used to generate O₂⁻, which reacted with XTT to produce a reddish product (470 nm). Addition of CuZnSOD to the reaction caused a dismutation of O₂⁻ and reduced OD₄₇₀ nm in a concentration-dependent manner (FIG. 19A). 2-ME prevented the reduction of absorbance at 470 nm in the presence of SOD (FIG. 19B), indicating that 2-ME inhibited SOD activity. The inhibition of human CuZnSOD, bovine CuZnSOD, and the E. coli MnSOD by 2-ME was concentration-dependent (IC₅₀≅20 μM, FIG. 19B). Furthermore, 2-ME (1-100 μM) showed little effect on xanthine oxidase (FIG. 19C), DNA polymerase α, or alkaline phosphatase (FIG. 19D), suggesting that inhibition of SOD by 2-ME is likely specific.

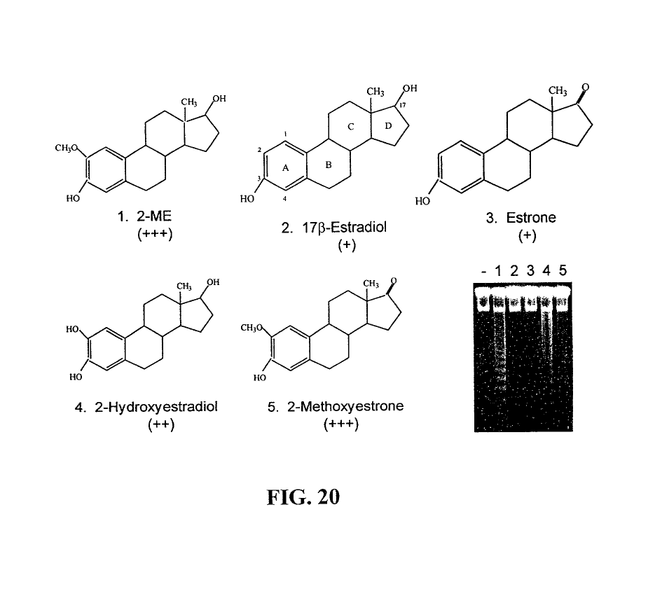

Four structurally related estrogen derivatives were compared with 2-ME to investigate the chemical basis for inhibition of SOD. As shown in FIG. 20, 2-ME (#1), 2-hydroxyestradiol (#4), and 2-methoxyestrone (#5), which have an —OH or —OCH₃ group at the 2-carbon, substantially inhibited SOD activity and induced DNA fragmentation. In contrast, 17β-estradiol (#2) and estrone (#3) lacking the 2-carbon modification showed minimal activity against SOD and caused little DNA fragmentation. It appeared that a 2—OH or 2-OCH₃ modification was important for inhibiting SOD and inducing apoptosis.

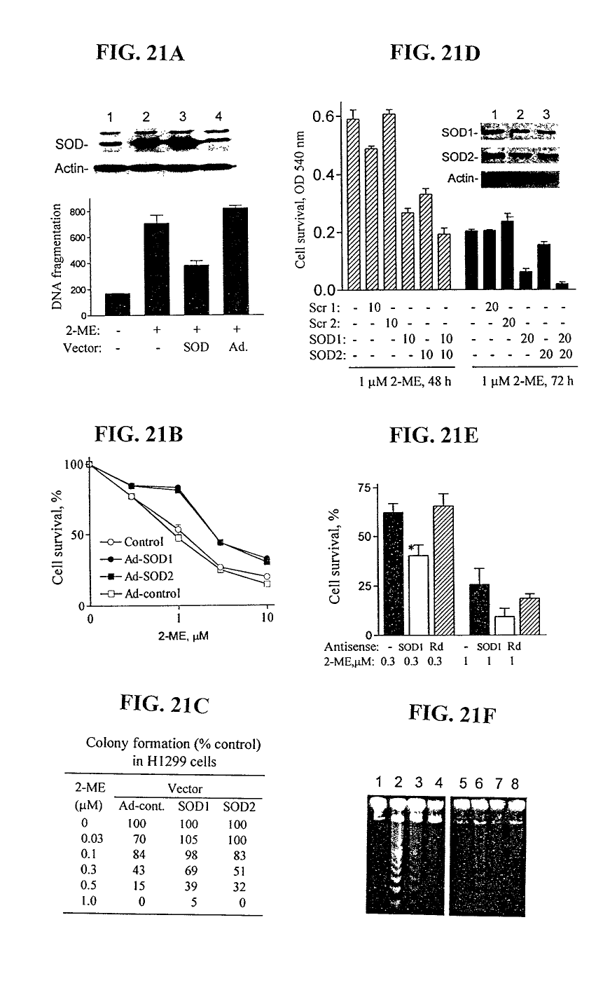

If inhibition of SOD were critical for the cytotoxic action of 2-ME, one would expect that a change in SOD expression would alter cellular sensitivity to 2-ME. When human ovarian cancer cells (A2008) was infected with an adenoviral vector containing full-length CuZnSOD sequence (Ad.CuZnSOD), substantial increases of SOD1 protein were detected at 24 h and 48 h (FIG. 21A, lanes 2 and 3). This overexpression led to a decrease of 2-ME-induced apoptosis, as evidenced by a reduction of nucleosomal DNA fragmentation (FIG. 21A). In contrast, infection with the control viral vector did not affect SOD expression (lane 4) or drug sensitivity. MTT assay also revealed a partial protection of the 2-ME-treated cells by transduction with Ad.CuZnSOD or Ad.MnSOD (FIG. 21B). Colony formation assay in another cell line (H1299) further confirmed this protective effect (FIG. 21C).

Synthetic antisense S-oligos against SOD1 and SOD2 were used to suppress SOD expression, and their effect on cellular sensitivity to 2-ME was then evaluated. As shown in FIG. 21D, incubation of A2008 cells with 10 μM each of the anti-SOD S-oligos for 48 h led to a moderate decrease of SOD1 and SOD2 protein. This was associated with an increase of 2-ME-induced cytotoxicity. A longer incubation (72 h) with 20 μM anti-SOD S-oligos resulted in a greater enhancement of 2-ME activity. Treatment with scrambled S-oligos did not alter the SOD protein levels nor did the cellular sensitivity to 2-ME change (FIG. 21D). Significant enhancement of the activity of 2-ME (0.3 μM, p=0.0059; 1 μM, p=0.0452) by anti-SOD1 S-oligo was also observed in H1299 cells (FIG. 21E). The role of SOD inhibition by 2-ME in causing apoptosis was further evidenced by experiments with antioxidants. When HL-60 cells were incubated with 1 μM 2-ME in the presence of the O₂⁻ scavenger ambroxol or antioxidant N-acetylcysteine, the 2-ME-induced apoptosis was significantly reduced (FIG. 21F). The protective effect of N-acetylcysteine was also observed in CLL cells using the MTT assay.

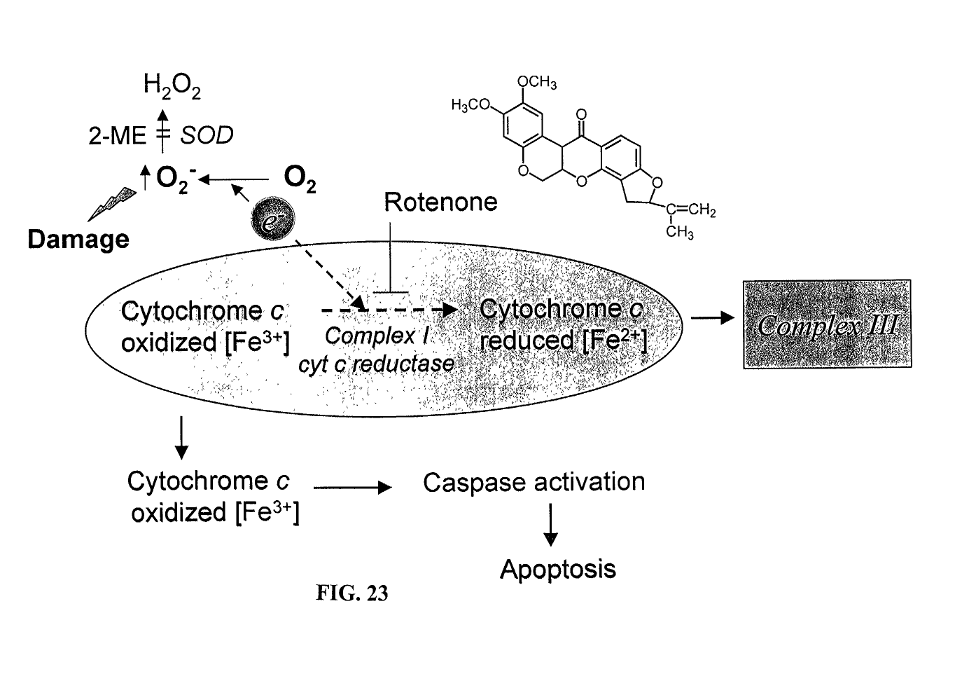

Because mitochondria are a major source of superoxide (O₂⁻) production, we reasoned that inhibition of SOD by 2-ME might cause mitochondrial damage due to free radical attack on the membrane phospholipids. To test this possibility, the integrity of mitochondrial membranes of 2-ME-treated cells was examined by measuring their ability to retain rhodamine-123, an fluorescent dye used to indicate the loss of mitochondrial transmembrane potential. Under the experimental conditions (rhodamine-123, 5 μg/ml, 30 min), the control HL-60 cells retained 70-100 arbitrary units of fluorescence (FIG. 23). During the first 6 h of 2-ME incubation, no obvious loss of dye retention was observed. However, significant loss of membrane integrity was seen after prolonged exposure to 2-ME (14 h), as evidenced by a shift of the fluorescence intensity to much lower levels (20-40 units). By 22 h, almost all the cells had lost their ability to retain rhodamine-123 in the mitochondria.

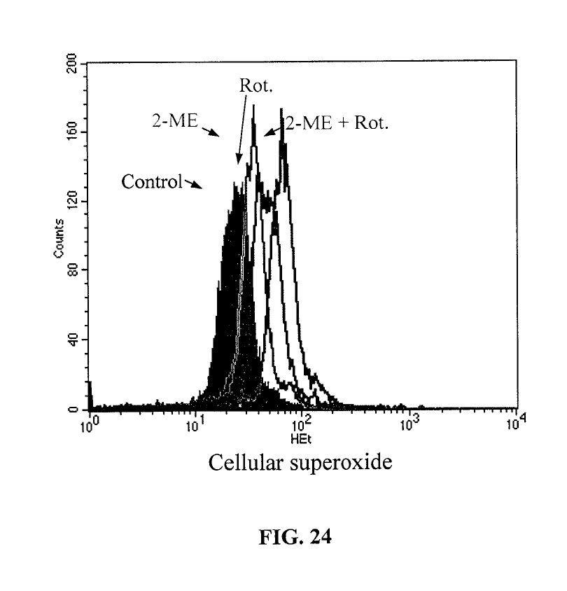

It is known that cytochrome c is normally located in mitochondria and its release to cytosol triggers apoptosis. Thus, the possibility that the mitochondrial membrane damage by 2-ME might result in the release of cytochrome c was tested. As shown in FIG. 24, no cytochrome c was present in cytosol during the first 10 h. However, significant amounts of cytosolic cytochrome c were detected at 12 and 14 h. This was in agreement with the time course of mitochondrial membrane damage. In separate experiments, it was observed that 2-ME-induced apoptosis occurred in the presence of cycloheximide (5-50 μg/ml) or actinomycin D (1 μg/ml), suggesting that induction of apoptosis by 2-ME did not require the synthesis of new RNA or protein. This is consistent with the mechanism of action of 2-ME in causing a free radical-mediated damage to mitochondria membrane and release of cytochrome c, which then activated the apoptotic cascade.

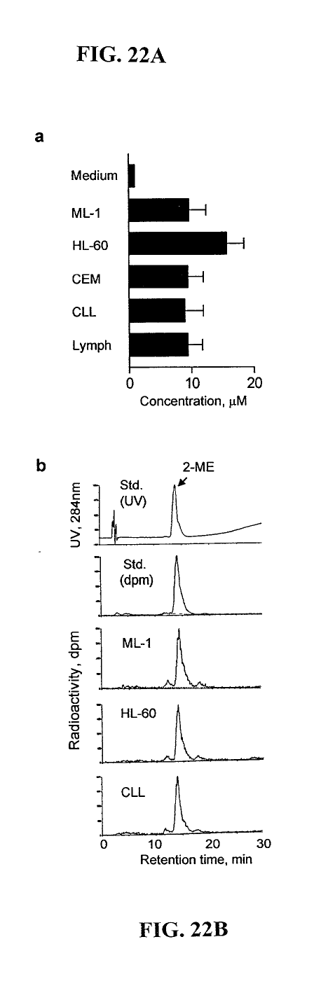

In the leukemia cell lines tested, 2-ME induced apoptosis at concentrations of 1-10 μM, whereas the IC₅₀ for SOD inhibition in vitro was approximately 20 μM. This apparent discrepancy was likely due to a concentrating uptake of 2-ME by the cells. To confirm this, we incubated leukemia cell lines, primary CLL cells, and normal lymphocytes with 1 μM [³H]2-ME, and determined the intracellular 2-ME concentrations. As shown in FIG. 22A, the drug was concentrated by approximately 10-fold in most cases during the 5-h incubation. HPLC analysis of the extracts from ML-1, HL-60 and CLL cells incubated with [3H]2-ME revealed no significant drug metabolites (FIG. 22B). 2-ME appeared as the only major peak (14.1 min) in all samples; there were two small peaks barely detectable at 11.8 and 17.6 min.

### Example 2

Based on our discovery that Superoxide dismutase (SOD) is a key target of 2-methoxyestradiol (2-ME) in causing apoptosis of cancer cells, we have further designed the following combination strategies to enhance anticancer activity.

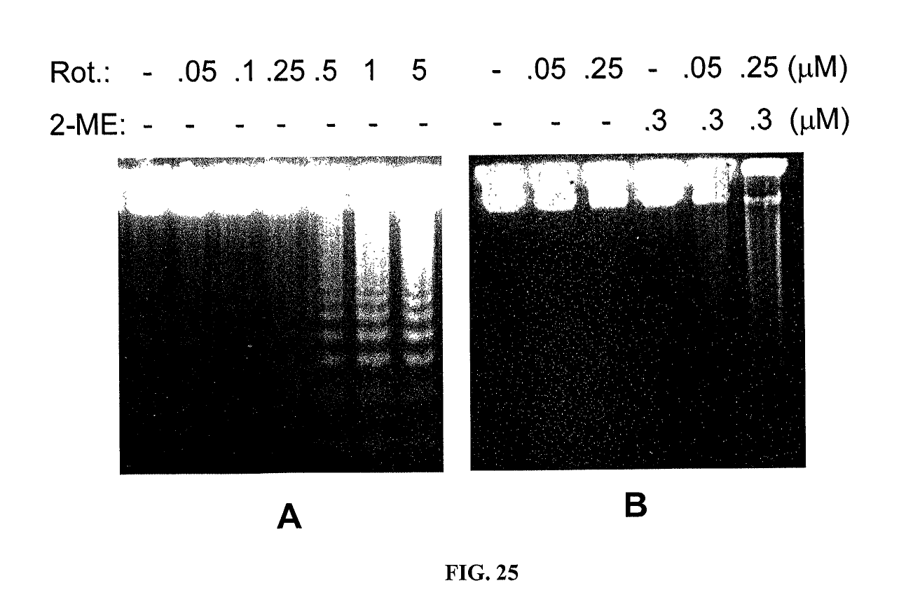

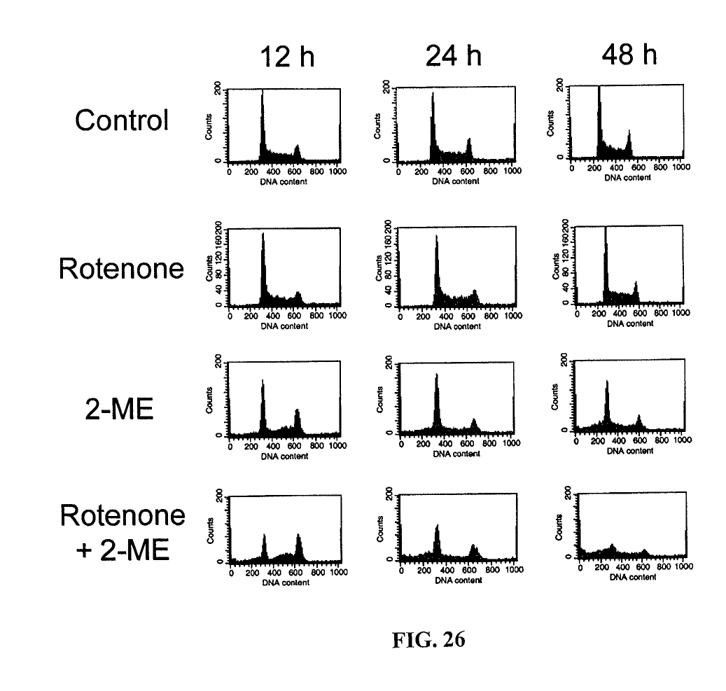

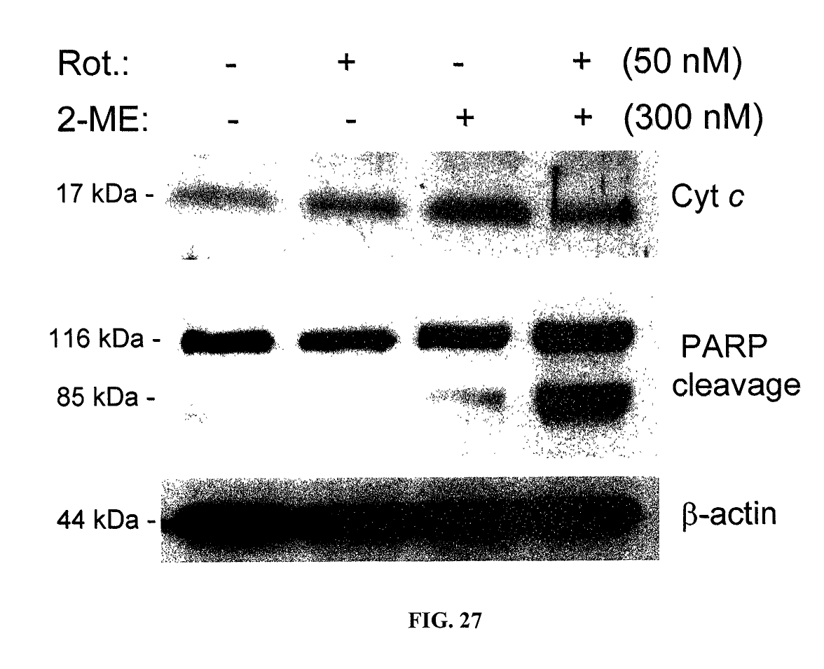

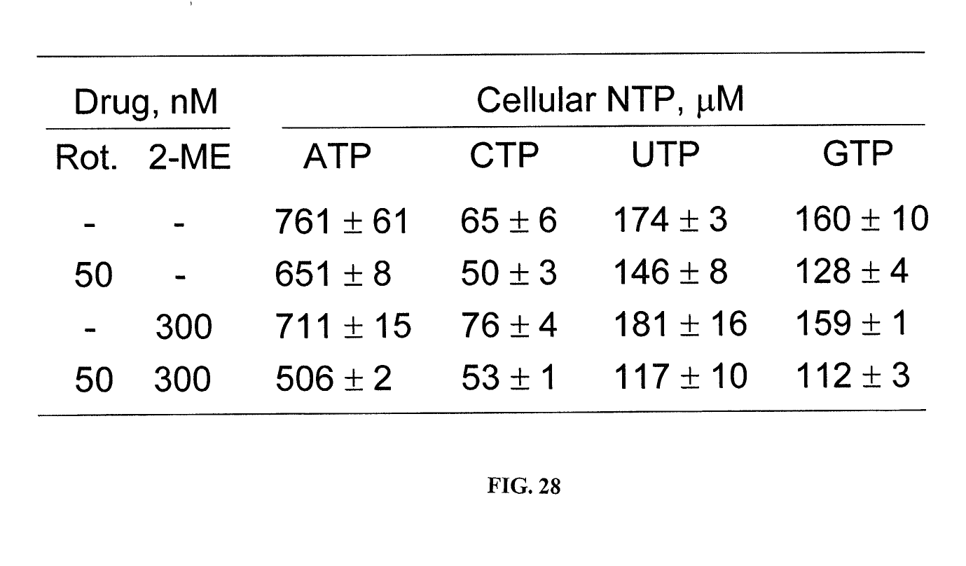

The first strategy involved a pharmacological approach to increase the generation of intracellular O₂⁻ by rotenone in combination with 2-ME to further block the elimination of O₂⁻, and thus enhance the free radical-mediated damage to the cells. This strategy is shown in FIG. 23. Rotenone, an inhibitor of mitochondrial enzyme complex I, inhibits the transport of electron and cause a leak of electron from complex I to form O₂⁻. We have established a flow cytometry-based method to quantitate cellular O₂⁻ contents. As shown in FIG. 24, incubation of HL-60 cells with 0.25 μM of 2-ME or rotenone led to an increase of cellular O₂⁻ Combination of both compounds caused a further O₂⁻ accumulation in the cells. When sub-toxic concentrations of 2-ME and rotenone were combined, substantial cytotoxic activity against HL-60 leukemia cells was observed. This synergistic effect was demonstrated by nucleosomal DNA fragmentation assay (FIG. 25), by flow cytometry analysis (FIG. 26), and by immunoblotting of PARP cleavage (FIG. 27). The killing of leukemia cells by the combination of 2-ME and rotenone was independent of cell cycle, as evidenced by depletion of cells from all phases of the cell cycle and the appearance of sub-G1 population (FIG. 26). This is consistent with the free radical-mediated damage to the mitochondrial membranes as a key mechanism of apoptosis induction. We further demonstrated that this synergistic effect was not due to change in cellular nucleotide pools (FIG. 28).

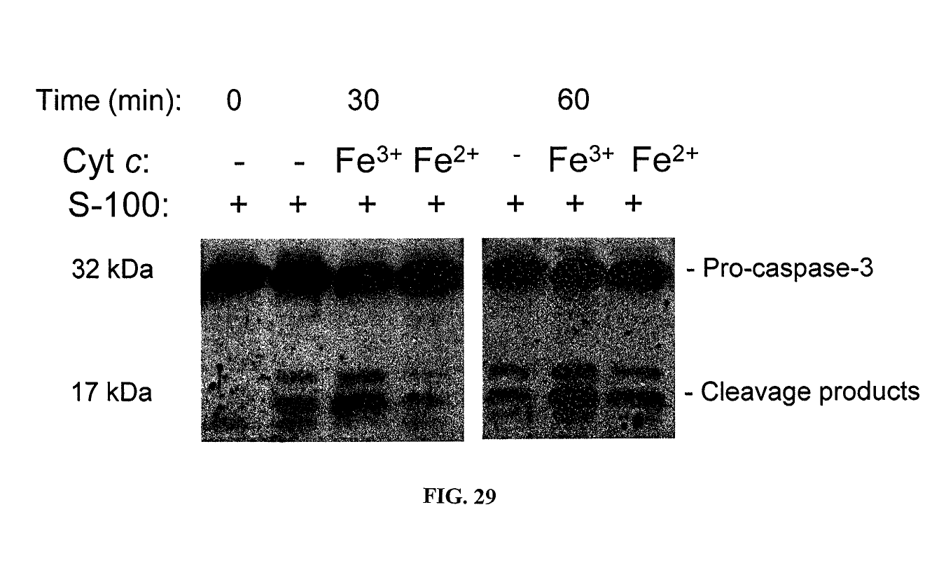

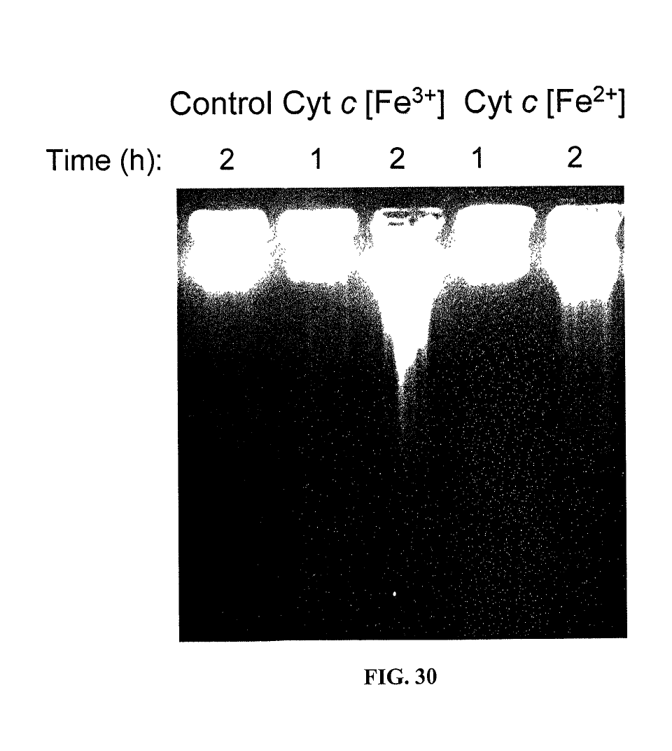

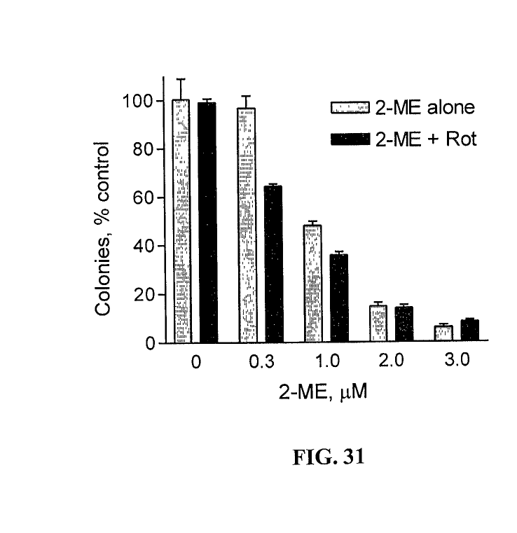

Importantly, we discovered that rotenone, by diverting the electron from complex I in the mitochondria to oxygen to form O₂⁻, also changed the redox status of cytochrome c, which remained in oxidized form (Fe³⁺). The oxidized form of cytochrome c was shown to be a more effective activator of apoptotic cascade, as demonstrated by the cleavage of caspase-3 in a cell free system (FIG. 29). Incubation of cytochrome c with isolated nuclei also showed that the oxidized form of cytochrome c was more effective in causing nucleosomal DNA fragmentation (FIG. 30). The enhancement of 2-ME activity by combination with rotenone was also observed in H1299 cells, using a separate end point (colony formation, FIG. 31).

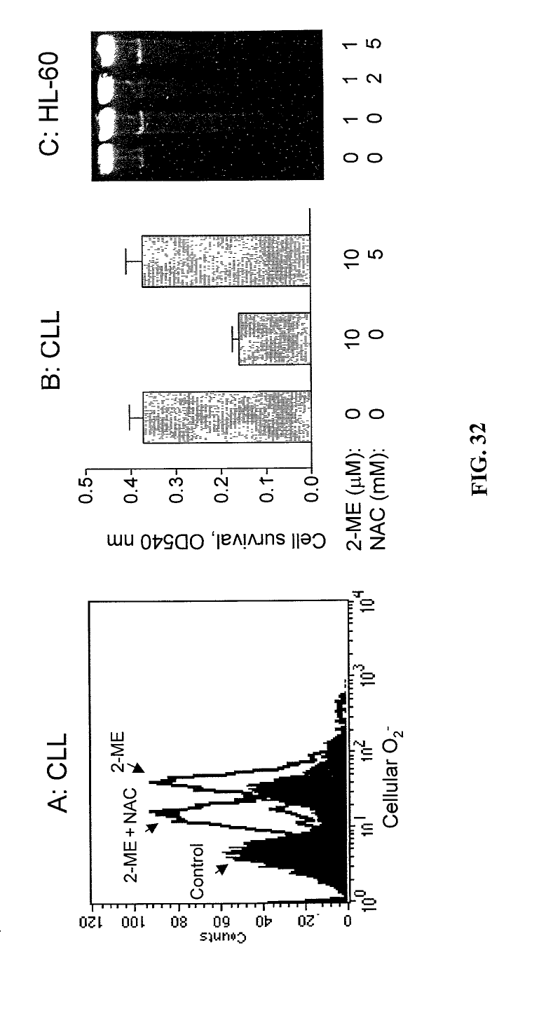

The important role of free radical in mediating the cytotoxic effect of 2-ME was further demonstrated by the use of an antioxidant N-acetylcysteine (NAC). As shown in FIG. 32, incubation of primary leukemia cells from a CLL patient with 2-ME in vitro caused a significant accumulation of cellular O₂⁻, and a loss of cell survival. Addition of NAC reduced the accumulation of cellular O₂⁻, and protected the cells from the toxic effect of 2-ME. These data support the conclusions that 2-ME causes cell death by a free radical-mediated mechanism as a consequence of SOD inhibition.

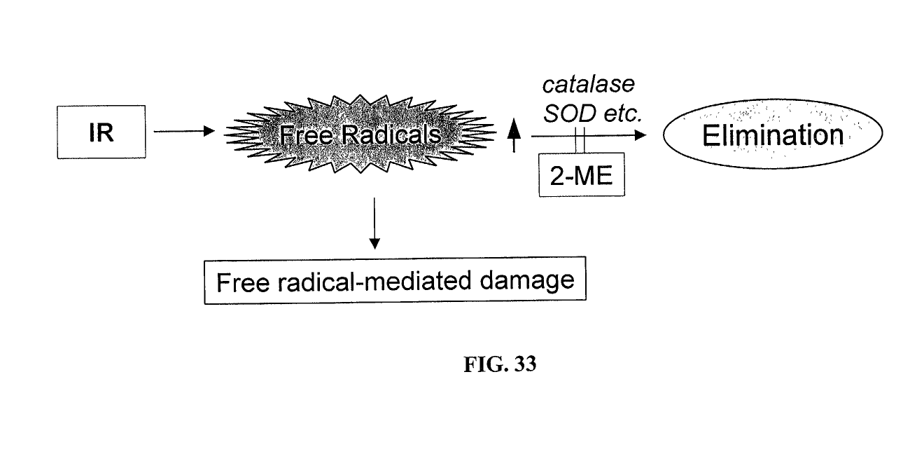

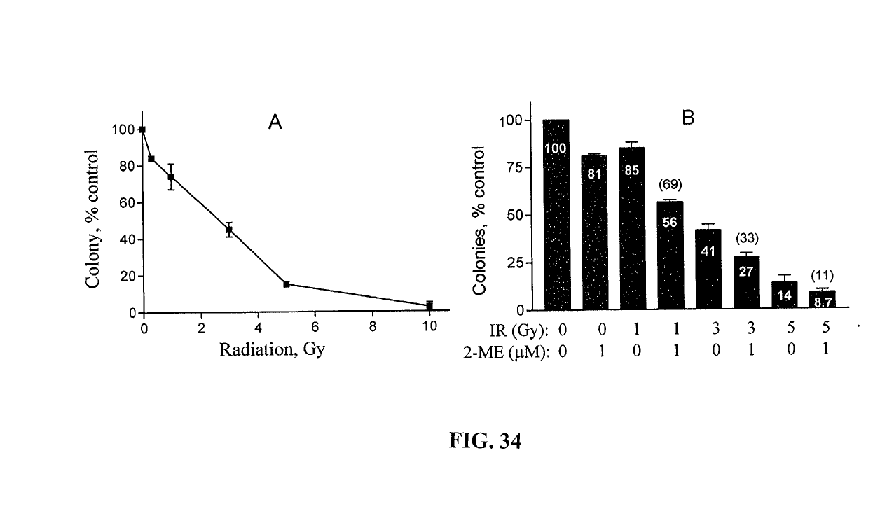

The second combination strategy to enhance anticancer activity involved combination of 2-ME with gamma rays (ionizing radiation, IR). The rationale for such a combination is shown in FIG. 33. It is known that ionizing radiation causes a generation of free radicals in the cells. Addition of 2-ME will cause an inhibition of SOD activity and thus compromise the cell's ability to cope with the free radicals generated by IR. This is expected to cause a greater killing of cancer cells. Indeed, the data from colony formation experiments in human lung cancer cells (H1299) support this hypothesis. FIG. 34 demonstrates the synergistic activity of 2-ME (1 μM, 24 h) and IR (various doses). The number inside each column indicates the observed surviving colonies. The number above each column shows the expected % survival, assuming that 2-ME and gamma radiation had an additive effect. In fact, the observed cell killing was greater than the expected additive effect under each combination condition.

### Example 3

2-Methoxylestradiol (2-ME) was combined with other agents for cancer therapeutics. These data support the original mechanism-based combination strategies and provide specific combinational therapies that are effective in cancer treatment. The data described herein were obtained in experiments with primary leukemia cells isolated from blood samples obtained from patients with chronic lymphocytic leukemia (CLL).

Combination with Sodium Arsenate.

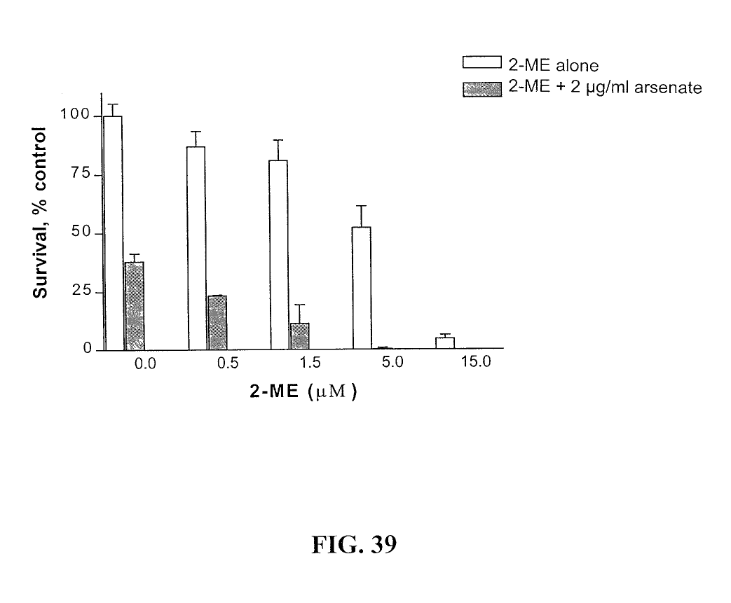

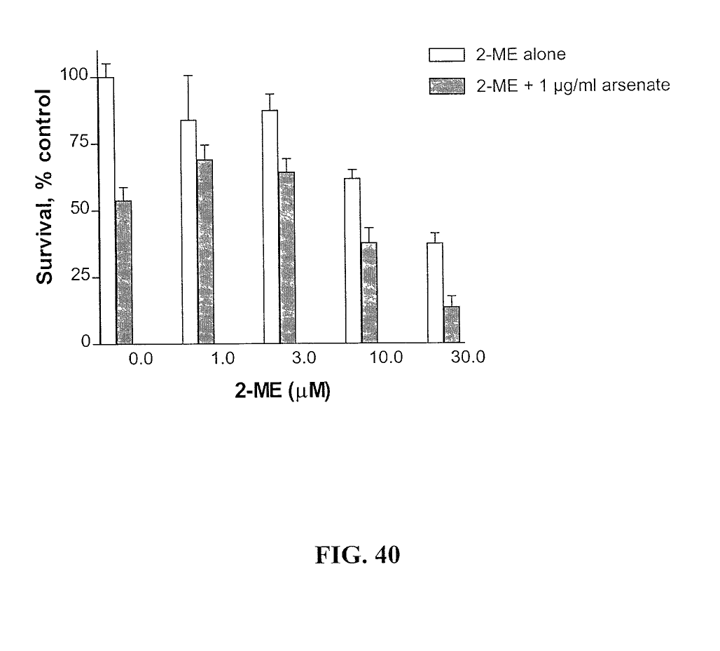

Arsenic trioxide is known to possess anticancer activity and has been approved for use in clinical treatment of certain type of leukemia. Induction of free radical generation in the cells is thought to contribute to its cytotoxic activity against cancer cells. It is hypothesized that combination of arsenate (to enhance cellular free radicals) and 2-ME (to inhibit the elimination of superoxide radicals) would increase their anticancer activity. As shown in FIGS. 39-40, the combination of arsenate and 2-ME substantially increased the cytotoxic activity against primary human leukemia cells from CLL patients in vitro. It should be noted that the CLL cells from these two patients were relatively sensitive to incubation with either 2-ME alone or arsenate alone. The combined effect appears to be additive.

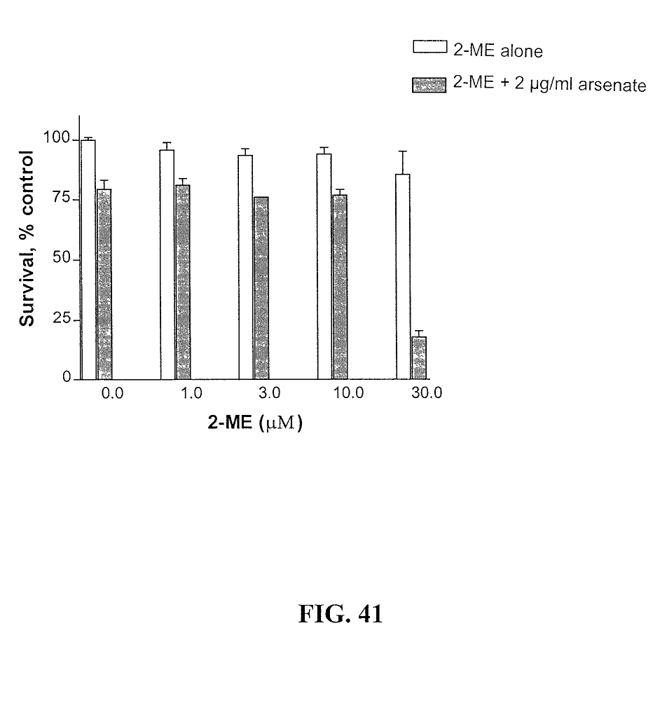

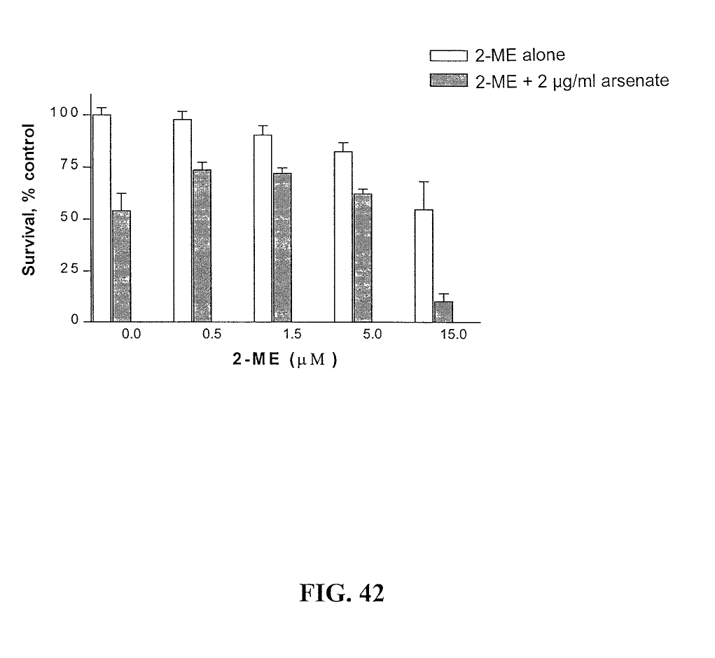

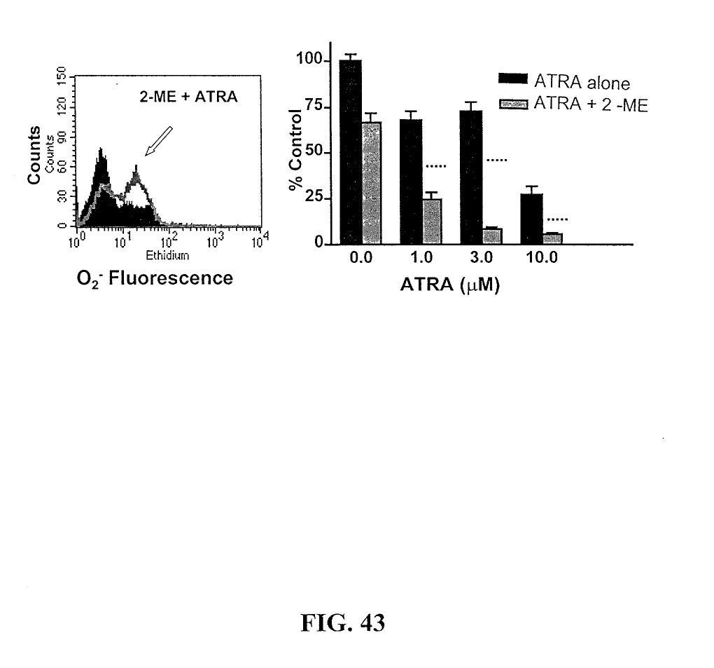

Importantly, leukemia cells isolated from difference patients show different sensitivity to 2-ME. The heterogeneous response to drug treatment in primary cancer cells from different patients requires new strategies to overcome drug resistance. Inventors tested if the combination of 2-ME with arsenate may overcome cell's resistance to 2-ME. Primary leukemia samples from two different CLL patients were identified to be resistant to 2-ME (up to 30 μM) in vitro. As shown in FIGS. 41-42, combination of arsenate with 2-ME (15 μM, FIG. 41; 30 μM, FIG. 42) caused significant loss of viability in the CLL cells, which were insensitive to either drug alone. Thus, such drug combination may have a potential to achieve therapeutic activity for patients who are insensitive to treatment with 2-ME alone.

Combination with Rituxan

Rituxan (or rituximab) is an antibody specific for CD20 antigen on B lymphocyte, and is used in the clinical treatment of certain B cell lymphoma. This agent was test for its combination effect with 2-ME in primary leukemia B cells (CLL) in vitro. Incubation of B CLL cells with various concentrations of Rituxan alone caused an increased expression of c-myc protein, but did not result in any significant killing of the CLL cells. There was only a moderate activity against CLL cells when Rituxan was combined with 2-ME at a fix ratio (2-ME/Rituxan=0.06). The results showed that Rituxan did not significantly enhance the activity of 2-ME either in cells sensitive to 2-ME alone or in cells resistant to 2-ME.

Combination with All-Trans Retinoic Acid (ATRA)

All-trans retinoic acid is an anticancer agent used in clinical treatment of cancer, especially hematological malignancies. Since retinoid derivatives have been shown to cause an increase of cellular free radicals, we tested if combination of 2-ME with ATRA may result in an enhanced anti-leukemia activity. As shown in FIG. 46, incubation of CLL cells with 2-ME and ATRA resulted in a significant increase of cellular superoxide content, and caused a synergistic activity against CLL cells. The dashed lines above the bars indicate the predicted additive effect. The observed anti-leukemia activity was clearly more than the additive effect of both compounds.

Conclusions

Combinations of 2-ME with arsenic compounds or retinoic acid derivatives will enhance anticancer activity and overcome drug resistance to 2-ME. Such combination can have clinical implications for cancer treatment.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Bindokas et al., J. Neurosci., 16, 1324-1336 (1996).

Bukowski, Semin. Oncol. 27:204-212, (2000).

Casppary et al., Mol. Pharmacol. 16, 256-260 (1979).

Cushman et al., J. Med. Chem. 38, 2041-2049 (1995).

D'Amato et al., Proc. Natl. Acad. Sci. USA. 91, 3964-3968 (1994).

Dionisi et al., Biochim Biophys Acta 403, 292-300 (1975).

Dragnev K H, et al., Oncologist, 5(5):361-8, (2000).

Eder, J., Steroid Biochem, 11 (1A):55-60, (1979).

Evans et al., JACS, 103, 5813 (1981).

Fang N, et al., Proc Natl Acad Sci USA. 95:3380-3384, (1998).

Flanagan et al., FEBS Letters. 431:285-286, (1998).

Fotsis et al., Nature, 368, 237-239 (1994).

Fridovich, Annu. Rev. Biochem. 64:97-112, (1995).

Fujimura et al., J. Neurosci., 19, 3414-3422 (1999).

Geissler et al., Blood, 94, 4230a, 1999.

Gutteridge et al., Biochem. J. 206, 605-609 (1982).

Halliwell et al., Free radicals in biology and medicine. Oxford University Press, New York (1985).

Hamana et al., Biochem. Int. 10, 301-309 (1985).

Heiden et al., Cell, 91, 627-637 (1997).

Lee et al., J. Biomed. Sci., 7(1):2-15, (2000).

Li et al., Cell, 91, 479-489 (1997).

Marklund et al., Cancer Res. 42, 1955-1961 (1982).

Marzo I. et al., Science, 281, 2027-2031 (1998).

Moreno-Manzano et al., J. Biol Chem. 275, 12684-12691 (2000).

Mukhopadhyay et al., Oncogene, 17, 241-246 (1998).

Nakamuri et al., Chem. Pharm. Bull., 10, 281 (1962).

Nishigaki et al: Photochem Photobiol. 70:228-235, (1999).

Oberley et al., Cancer Res 39, 1141-1149 (1979).

Oppolzer and Roberts, Helv. Chim. Acta., 63, 1703, (1980).

Pervaiz et al., FEBS Lett. 459, 343-348 (1999).

Rigo et al., Biochem. Biophy. Res. Commun. 63, 1013-1018 (1975).

Robins et al: Cancer Chemother Pharmacol, 43:409-414, (1999).

Rousselot et al., Blood, 94:4457a, (1999).

Shulyakovskaya et al., Biochem. Biophys. Res. Commun. 95, 581-587 (1993).

Soignet et al., Blood, 94:1247a, (1999a).

Soignet et al., Cancer Chemother. Pharmocol., 44:471-421, (1999b).

Sunagawa et al., Chem. Pharm. Bull., 9, 81, (1961).

The Merck Index, 11th Ed., Merck & Co., Inc., Rahway, N.J. U.S.A. (1989), pp. 583-584

U.S. Pat. No: 6,191,123

U.S. Pat. No: 4,578,770

U.S. Pat. No: 4,596,792

U.S. Pat. No: 4,599,230

U.S. Pat. No: 4,599,231

U.S. Pat. No: 4,601,903

U.S. Pat. No: 4,608,251

Ukeda et al., Anal. Biochem. 251, 206-209 (1997).

Van Driel et al., J. Histochem. Cytochem. 47, 575-582 (1999).

Van Tamelen et al., Tetrahedran 14, 8-34 (1961).

Wiernik et al., Blood, 94:22893a, (1999).

Young et al., New Engl J. Med. 305,139-153(1985).

Zwacka et al., Hum. Gene Ther., 9, 1381-1386 (1998).

What is claimed is:

1. A method of killing a cell comprising:
a) contacting said cell with a first composition comprising an agent that increases intracellular O₂⁻; and
b) contacting said cell with a second composition comprising 2-methoxyestradiol.

2. The method of claim 1, wherein said cell is a cancer cell.

3. The method of claim 2, wherein said cancer cell is derived from a solid tumor.

4. The method of claim 2, wherein said cancer cell is a leukemia cell.

5. The method of claim 1, wherein said cell is a human cell.

6. The method of claim 1, wherein said compound that increases intracellular O₂⁻ is rotenone.

7. The method of claim 1, wherein said compound that increases intracellular O₂⁻ comprises bleomycin.

8. The method of claim 1, wherein said compound that increases intracellular O₂⁻ comprises daunorubicin.

9. The method of claim 1, wherein said compound that increases intracellular O₂⁻ comprises epirubcin.

10. The method of claim 1, wherein said agent that increases intracellular O₂⁻ comprises TNF-alpha.

11. The method of claim 1, wherein said agent that increases intracellular O₂⁻ comprises heat.

12. The method of claim 1, wherein said agent that that increases intracellular O₂⁻ comprises an arsenate.

13. The method of claim 1, wherein said agent that that increases intracellular O₂⁻ comprises a retinoic acid derivative.

14. The method of claim 1, wherein the administration of said first composition and said second composition is substantially concurrent.

15. The method of claim 1, wherein the administration of said first composition is subsequent to the administration of said second composition.

16. The method of claim 1, wherein the administration of said first composition is prior to the administration of said second composition.

17. The method of claim 1, wherein said first and said second compositions are combined in a single formulation.

18. A method of treating cancer comprising administering to a host a composition comprising 2-methoxyestradiol and an agent that increases intracellular O₂⁻.

19. The method of claim 18, wherein said agent that increases intracellular O₂⁻ is rotenone.

20. The method of claim 18, wherein said agent that increases intracellular O₂⁻ comprises bleomycin.

21. The method of claim 18, wherein said agent that increases intracellular O₂⁻ comprises daunorubicin.

22. The method of claim 18, wherein said agent that increases intracellular O₂⁻ comprises epirubcin.

23. The method of claim 18, wherein said agent that increases intracellular O₂⁻ comprises TNF-alpha.

24. The method of claim 18, wherein said agent that increases intracellular O₂⁻ comprises heat (hyperthermia).

25. The method of claim 18, wherein said agent that that increases intracellular O₂⁻ comprises an arsenate.

26. The method of claim 18, wherein said agent that that increases intracellular O₂⁻ comprises a retinoic acid derivative.

27. The method of claim 18, wherein said host is a human.

28. The method of claim 18, wherein the administration of said first composition and said second composition is substantially concurrent.

29. The method of claim 18, wherein the administration of said first composition is subsequent to the administration of said second composition.

30. The method of claim 18, wherein the administration of said first composition is prior to the administration of said second composition.

31. The method of claim 18, wherein said first and said second compositions are contained within a pharmaceutically acceptable composition.

32. The method of claim 31, wherein said pharmaceutically acceptable composition includes a pharmaceutically acceptable carrier.

33. The method of claim 31, wherein said pharmaceutical composition is formulated for oral administration.

34. The method of claim 31, wherein said pharmaceutical composition is formulated for parenteral administration.

35. The method of claim 31, wherein said pharmaceutical composition is formulated for administration by injection.

36. The method of claim 18, wherein said host has cancer.

37. The method of claim 36, wherein said cancer is a solid tumor.

38. The method of claim 36, wherein said cancer is a leukemia.

39. The method of claim 18, wherein said first and said second compositions are combined in a single formulation.

40. A composition comprising 2-methoxyestradiol and a second compound that increase intracellular O₂⁻.

41. The composition of claim 40, wherein said compound that increases intracellular O₂⁻ comprises rotenone.

42. The composition of claim 40, wherein said compound that increases intracellular O₂⁻ comprises bleomycin.

43. The composition of claim 40, wherein said compound that increases intracellular O₂⁻ comprises daunorubicin.

44. The composition of claim 40, wherein said compound that increases intracellular O₂⁻ comprises epirubicin.

45. The composition of claim 40, wherein said agent that that increases intracellular O₂⁻ comprises an arsenate.

46. The composition of claim 40, wherein said agent that that increases intracellular O₂⁻ comprises a retinoic acid derivative.

47. The composition of claim 40, wherein said composition is a pharmaceutically acceptable composition.

48. The composition of claim 40, wherein said compound that increases intracellular O₂⁻ comprises tumor necrosis factor-alpha.

49. The composition of claim 48, wherein said pharmaceutically acceptable composition includes a pharmaceutically acceptable carrier.

50. The composition of claim 48, wherein said pharmaceutical composition is formulated for oral administration.

51. The composition of claim 48, wherein said pharmaceutical composition is formulated for parenteral administration.

52. The composition of claim 48, wherein said pharmaceutical composition is formulated for administration by injection.


SEQUENCE LISTING (6 sequences)

SEQ ID NO: 1 - DNA, 27 bp, Artificial Sequence
  Description of Artificial Sequence  Synthetic Primer
acgcacacggccttcgtcgccataact

SEQ ID NO: 2 - DNA, 25 bp, Artificial Sequence
  Description of Artificial Sequence  Synthetic Primer
gcacactgcccggctcaacatgctg

SEQ ID NO: 3 - DNA, 23 bp, Artificial Sequence
  Description of Artificial Sequence  Synthetic Primer
acgaaggccgtgtgcgtgctgaa

SEQ ID NO: 4 - DNA, 23 bp, Artificial Sequence
  Description of Artificial Sequence  Synthetic Primer
accacaagccaaacgacttccag

SEQ ID NO: 5 - DNA, 24 bp, Artificial Sequence
  Description of Artificial Sequence  Synthetic Primer
ccatcaatgaccccttcattgacc

SEQ ID NO: 6 - DNA, 23 bp, Artificial Sequence
  Description of Artificial Sequence  Synthetic Primer
gaaggccatgccagtgagcttcc